(19) United States
(12) Patent Application Publication
(10) Pub. No.: US 2026/0031197 A1
(43) Pub. Date: Jan. 29, 2026

(54) MESSAGE PASSING GRAPH NEURAL NETWORK WITH VECTOR-SCALAR MESSAGE PASSING AND RUN-TIME GEOMETRIC COMPUTATION

(71) Applicant: Microsoft Technology Licensing, LLC, Redmond, WA (US)

(72) Inventors: Tong WANG, Beijing (CN); Bin SHAO, Beijing (CN); Tieyan LIU, Beijing (CN)

(73) Assignee: Microsoft Technology Licensing, LLC, Redmond, WA (US)

(21) Appl. No.: 19/099,892

(22) PCT Filed: Oct. 21, 2022
(86) PCT No.: PCT/CN2022/126834

§ 371 Date: Jan. 30, 2025

(51) Int. Cl. G16C 20/70; G06N 3/042; G16C 10/00; G16C 20/30

(52) U.S. Cl. CPC G16C 20/70; G06N 3/042; G16C 10/00; G16C 20/30

(57) ABSTRACT

A computing system is provided, which receives a molecular graph at a message passing graph neural network (MPGNN), and produces scalar embeddings representing features of nodes and edges of the graph and vector embeddings representing geometric relationships of the graph. The system processes the scalar embeddings via a vector scalar interactive message passing mechanism of a message passing sub-block of the

MPGNN to generate and pass scalar information from the scalar embeddings to an embedding space containing the vector embeddings. The system updates the vector embeddings based on the embedding space containing the scalar information and the vector embeddings. The system updates the scalar embeddings based on run-time geometry calculations of the geometric relationships encoded in the vector embeddings. The system computes an updated molecular graph based on the updated scalar and vector embeddings and outputs a target molecular property value based on the updated molecular graph.

### BACKGROUND

In the field of computational chemistry, computer-based techniques have been developed to predict molecular properties through computer simulations. These molecular properties can have a wide-ranging impact on the appearance and function of a molecule or material, and thus are of keen interest in a wide variety of fields. For example, in the field of drug design, changes in molecular properties can affect the efficacy of a drug. In the field of drug discovery, molecular properties can affect the potential for a material found in nature to be used for therapeutic purposes. In the field of quantum chemistry, quantum-mechanical calculation of electronic contributions to physical and chemical properties of molecules and materials is a fundamental area of inquiry. As discussed below, opportunities remain for improvements in computational methods for predicting molecular properties, which would have application beyond the field of computational chemistry.

### SUMMARY

To address the issues discussed herein, computerized systems and methods are provided. In one aspect, the computerized system includes a processor that receives a molecular graph at a message passing graph neural network (MPGNN), and produces scalar embeddings representing features of nodes and edges of the graph and vector embeddings representing geometric relationships of the graph. The system processes the scalar embeddings via a vector scalar interactive message passing mechanism of a message passing sub-block of the MPGNN to generate and pass scalar information from the scalar embeddings to an embedding space containing the vector embeddings. The system updates the vector embeddings based on the embedding space containing the scalar information and the vector embeddings. The system updates the scalar embeddings based on run-time geometry calculations of the geometric relationships encoded in the vector embeddings. The system computes an updated molecular graph based on the updated scalar and vector embeddings and outputs a target molecular property value based on the updated molecular graph.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows a schematic view of a computing system including a message passing graph neural network (MPGNN).

FIG. 2 shows a detail view of the MPGNN of the computing system of FIG. 1.

FIG. 3 shows a schematic view of a runtime angle calculation of a direction unit vector and relational position bond angle vector, and a run time dihedral angle calculation, performed by the MPGNN of the computing system of FIG. 1.

FIG. 4 shows interatomic distance distributions of MD simulations driven by the system of FIG. 1 and Density Functional Theory (DFT).

FIG. 5 shows an energy landscape of Chignolin and evaluations of energy prediction by the system of FIG. 1, an Equivariant Transformations (ET) model, and a Molecular Mechanics (MM) model.

FIG. 6 shows a visualization and model interpretability of the system of FIG. 1.

FIG. 7 is a table illustrating the performance of the system of FIG. 1, which shows mean absolute errors (MAE) of energy (kcal/mol) and forces (kcal/mol/° A) for 7 small organic molecules in MD17.

FIG. 8 is a table illustrating the performance of the system of FIG. 1, which shows MAE of energy (kcal/mol) and forces (kcal/mol/° A) for 10 small organic molecules in rMD17.

FIG. 9A shows a general schematic view of a workflow of scalar and vector embeddings of an vector scalar interactive message passing (ViS-MP) mechanism of the system of FIG. 1.

FIG. 9B shows a detail schematic view of an edge fusion graph attention module of the vector scalar interactive message passing mechanism of FIG. 9A.

FIG. 10 shows a schematic view of potential energy surfaces (PES) explored by molecular dynamics simulation driven by the system of FIG. 1 and DFT for the 7 molecules in MD17.

FIG. 11 shows a graph of time consumption of molecular dynamics simulations driven by the system of FIG. 1 and DFT.

FIG. 12 is a table illustrating the performance of the system of FIG. 1, which shows MAE of 12 kinds of molecular properties on QM9.

FIG. 13 shows an ablation study of the system of FIG. 1 on aspirin in MD17 dataset.

FIGS. 14-16 show a flowchart of a computerized method according to one example implementation of the present disclosure.

FIG. 17 shows an example computing environment according to which the embodiments of the present disclosure may be implemented.

### DETAILED DESCRIPTION

Computer-based techniques have been developed to predict molecular properties through computer simulations. For example, Density Functional Theory (DFT) is a powerful and widely used quantum physics calculation technique that can in many cases accurately predict various molecular properties such as energy and forces of molecules, the shape of molecules, etc. However, DFT is time-consuming and computationally intensive, often taking up to several hours even for a single model of a simple molecule on a conventional processor. For many complex systems, computing exact DFT solutions is not practical on current hardware. This currently presents a barrier to predicting molecular properties.

Recently, neural network models have been developed for application in the field of molecular dynamics simulation. Although the accuracy of these models has been improving, as discussed below, these models generally suffer from the drawback of having high computational costs. Accordingly, the widespread application of such neural network models in molecular dynamics simulations faces a challenge.

Molecular dynamics (MD) models compute potential energies and resultant atomic forces at each atom of a molecular system as the atoms change physical position over a simulation time period, to thereby describe kinetic and thermodynamic properties of the molecular system. MD is widely used in the physical, chemical, biological and pharmaceutical fields. Ab initio MD simulations such as those driven by DFT can accurately calculate energy and forces, although with a high computational cost, limiting the application of such techniques to large molecular systems and long simulations as discussed above. By contrast, classical MD simulations employing empirical force fields can achieve fast simulation results for large systems, but suffer from the drawback that they cannot capture the quantum effect caused by electron movement and generally the parameters of the force fields computed within such simulations are not transferable.

In recent years, deep learning (DL) has demonstrated its powerful ability to learn from raw data without any handcrafted features in many fields, and DL models that compute potential energies of molecular systems have attracted more and more attention. However, an inherent drawback of deep learning is that it requires large amounts of data, and this has become a barrier to its wider application in more scenarios. To alleviate the dependency on data for DL models that compute potential energies, inductive bias of symmetry can be incorporated into the design of a neural network, in a subfield termed geometric deep learning (GDL). Here, symmetry describes the conservation of physical laws, i.e., physical properties that remain unchanged in spite of transformations performed on the underlying data, such as translations or rotations. Due to these limitations, GDL can be extended to only to limited data scenarios without the need for data augmentation.

Within GDL, Equivariant Graph Neural Networks (EGNN) have been proposed to model molecular geometry. One type of EGNN for computing energy potentials achieves equivariance based on group representation theory, utilizing high-order geometric tensors. Although this approach makes use of geometric information, certain operations such as the Clebsch-Gordan product (CG-product) usually lead to ultra-large computational overheads at an intolerable computational scale, which severely inhibits this approach from being applied to large molecules in practice. To alleviate this and improve the modeling of directional information, another approach has utilized vector embedding and scalarized angular representations via inner products of the vector embedding itself, thereby capturing equivariance in the model. Models such as these have encoded angle and dihedral information of the molecular system explicitly, which has somewhat lowered the computational cost by avoiding CG-product operations. However, even these models suffer from relatively high computational cost and leave room for improvement of in terms of accuracy. Further, these models suffer from the drawback that they are not robust to various conformations of a molecular system during molecular dynamics simulations. These models appear to fail to effectively utilize such geometric information in message passing, and this may limit their performance.

To address these issues, a computing system configured to execute a message passing graph neural network MPGNN is provided, which utilizes directional information to achieve high accuracy at low computational costs. As discussed below, embodiments of the computing system with MPGNN described herein have outperformed state-of-the-art approaches for molecules in the Molecular Dynamics 17 (MD17) Dataset and revised rMD17 Dataset and have achieved superior prediction scores for 11 of 12 quantum properties on the Quantum Machine 9 (QM9) Dataset. In addition, simulation results show that the embodiments described herein have exhibited the potential technical benefit of being able to scale to protein molecules containing hundreds of atoms, while achieving ab initio accuracy (i.e., a level of accuracy close to the ground truth data the model is trained on) without molecular segmentation. Evaluations and case studies discussed below demonstrate that these embodiments can potentially efficiently explore the conformational space of small and large molecules alike, while providing reasonable interpretability to map geometric representations to molecular structures.

At a high level, within the MPGNN a Run-time Geometry Calculation (RGC) function is executed to extract and encode angular and dihedral information with linear computational complexity, significantly accelerating model training and inference as well as reducing the memory consumption. In addition, a vector scalar interactive message passing mechanism is adopted to effectively utilize geometric information by combining vectorial hidden representations with scalar hidden representations, in an equivariant manner. By incorporating these two modules, the MPGNN can achieve high computational efficiency with sufficient utilization of geometric information. When comprehensively evaluated on benchmarks, the embodiments of the MPGNN described herein outperform state-of-the-art algorithms on all molecules in the MD17 and revised MD17 datasets and show superior performance on the QM9 dataset, indicating the powerful capability of molecular geometric representation. Next, ab initio molecular dynamics simulations (AIMD) for each molecule on MD17 driven by the MPGNN trained only with 0.7% of the data are discussed. The highly consistent interatomic distance distributions and the explored potential energy surfaces between AIMD and quantum simulation illustrate that the MPGNN of the subject disclosure is data efficient and can perform simulations with high fidelity. To further explore the scalability of the MPGNN to large molecules, a full-atom MD dataset for the simplest protein Chignolin at DFT level that consists of 9543 different conformations of the 166-atom protein derived from replica exchange molecular dynamics and calculated by DFT was built. This is believed to be the first MD dataset for real-world full-atom proteins at the DFT level. When evaluated on this dataset, the MPGNN of the present disclosure also achieved the superior performance compared with other deep learning models for predicting potential energies and empirical force fields. In addition, the MPGNN is shown to exhibit reasonable interpretability to map geometric representation to molecular structures.

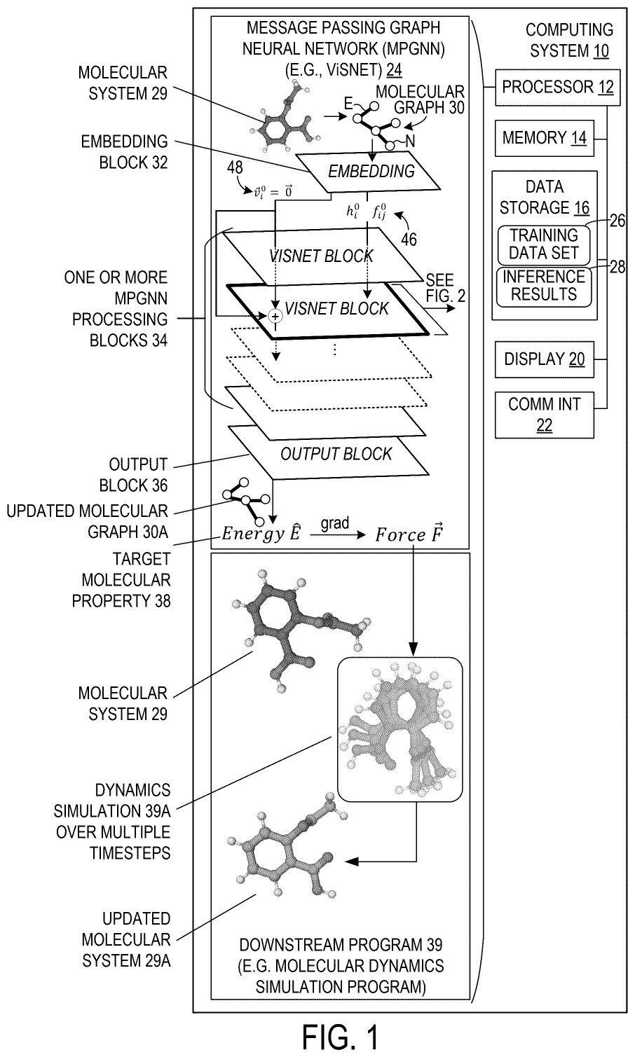

FIG. 1 illustrates a computing system 10 including one or more processors 12 operatively coupled via a data bus 14 to an associated memory 16, data storage 18, display 20, and communication interface 22. The one or more processors 12 are configured to execute a message passing graph neural network (MPGNN) 24 in a training phase to train the MPGNN 24 on a training data set 26, and to execute the trained MPGNN 24 during an inference phase and thereby produce inference results 28. Both the training data set and the inference results may be stored in data storage 16.

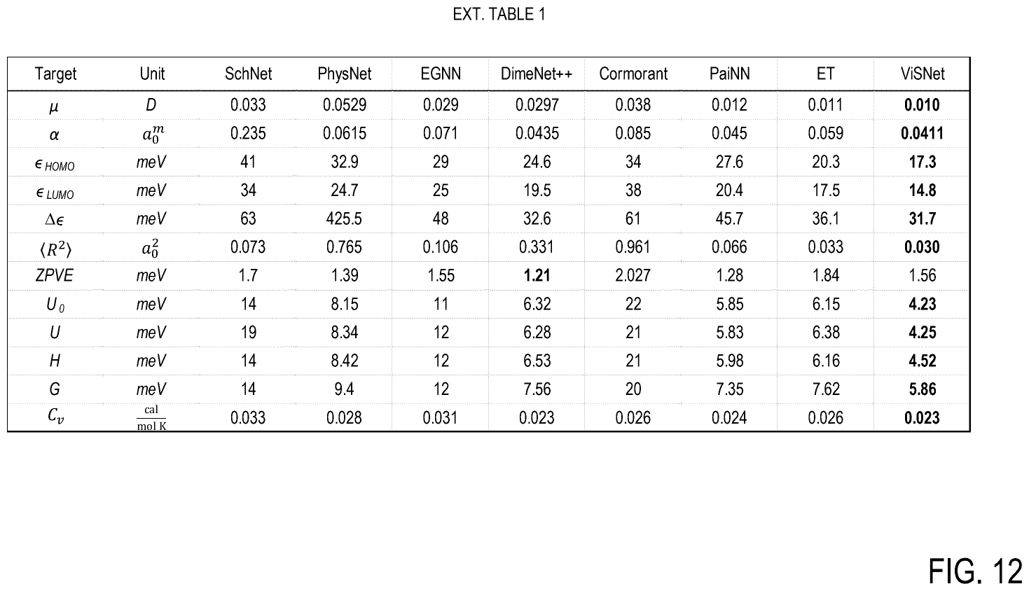

During the training phase, i.e., prior to the inference phase, the one or more processors are configured to train on training data set 26, which includes multiple molecular graphs 30 for different conformation geometries of a molecular system 29, and a respective ground truth value for a target molecular property 38 for each molecular graph 30. As discussed below in relation to FIG. 12, a variety of target molecular properties 38 may be used. For example, the target molecular property 38 can be an energy parameter, a force parameter, or a dipole moment. In one example, the target molecular properties 38 may be predicted in tandem by the MPGNN 24. The ground truth value is typically computed via density functional theory (DFT), although other types of numerical solvers may be used if desired.

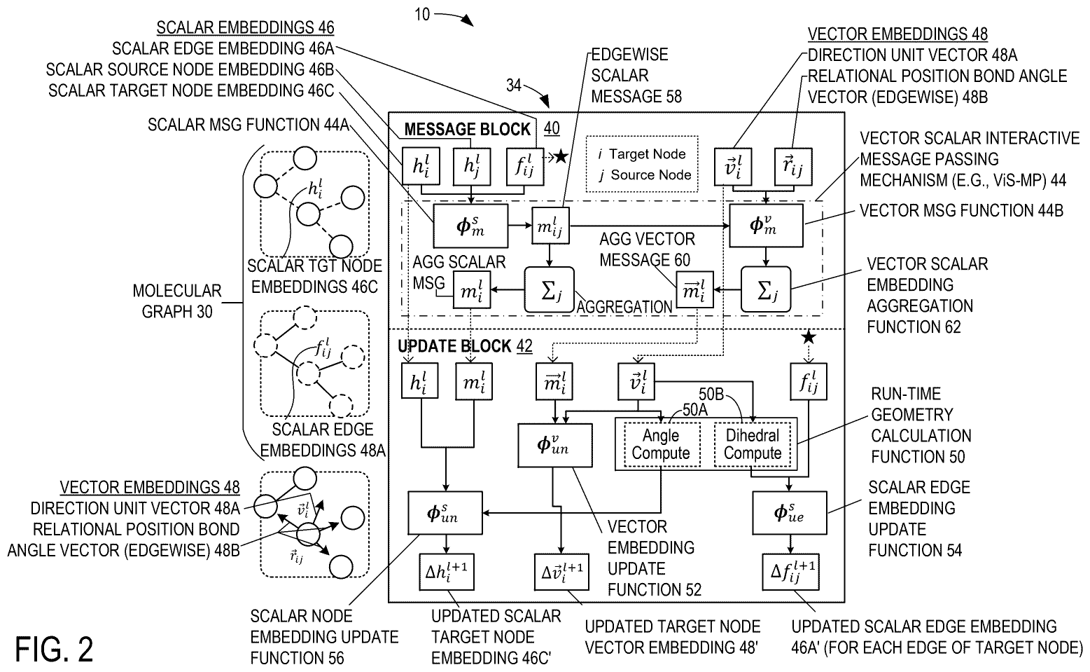

As illustrated in FIG. 1, the MPGNN 24 includes an embedding block 32, one or more MPGNN processing blocks 34, and an output block 36. A detail view of the internal structure of each MPGNN processing block 34 is shown in FIG. 2. Turning briefly to FIG. 2, each MPGNN processing block 34 includes a respective message passing sub-block 40 and a respective update sub-block 42. The message passing sub-block 40 is configured with a vector scalar interactive message passing mechanism 44.

Returning to FIG. 1, during the inference phase, the one or more processors 12 are configured to execute the trained MPGNN 24, and feed inference time input to the MPGNN. To this end, the one or more processors are further configured to receive a molecular graph 30 of the molecular system 29 as input to the MPGNN at inference time. The molecular graph 30 input at inference time is typically a molecule of the same type on which the MPGNN 24 was trained during the training phase, but having a different conformation geometry than the training examples included in the training data set 26. The molecular graph 30 typically includes nodes N connected by edges E. The nodes N represent atoms and the edges E represent interatomic bonds in the molecular system 29.

The one or more processors 12 are further configured to process the molecular graph 30 using the embedding block 32, to thereby produce scalar embeddings 46 encoding scalar information describing features of the nodes N and edges E and vector embeddings 48 representing geometric relationships among the nodes and edges of the molecular graph, as described in more detail below.

Referring briefly to FIG. 2, the scalar embeddings 46 include scalar node embeddings 46B, 46C and scalar edge embeddings 46A. The scalar node embeddings 46B, 46C encode a type of an atom represented by each node in the molecular graph 30. Scalar target node embeddings 46C (hᵢ) encode such information for the target node i and scalar source node embeddings 46B (hⱼ) encode such information for each source node j connected by an edge ij (i.e., molecular bond) to the target node i. The type of the atom may be indicated as a chemical symbol, or using another encoding scheme for example. The scalar edge embeddings 46A (fᵢⱼ) encode an interatomic distance represented by each edge E in the molecular graph 30. Other scalar information, such as the types of bonds, functional group labels, etc. may also be included if desired. These are exemplary scalar information types including in these embeddings, and it will be appreciated that other scalar information may also be encoded in the scalar embeddings 46. The vector embeddings 48 encode geometric information including a direction unit vector 48A for each node N and a relative position bond angle vector 48B for each of a plurality of node pairs connected by a bond (i.e., edge in the molecular graph 30) in the molecular graph. Other geometric information may also be included in the vector embeddings 48. It will be appreciated that the geometric information describes the angles of the molecular bonds between atoms, while the scalar information describes the distance between atoms and the types of atoms included in the molecular graph 30.

During inference, initial values for the molecular graph are provided as input, and then the MPGNN processing blocks 34 each update a prediction of changes in geometry of the molecular graph over a discrete time period. Thus, it will be appreciated that processing the molecular graph using the embedding block includes encoding, via the embedding block, initial values for the scalar node embeddings, the scalar edge embeddings, and the vector embeddings for the molecular graph. These initial values are then acted upon and updated by MPGNN processing blocks 34 arranged in successive layers during inference.

To that end, referring to FIG. 2, the one or more processors 12 are further configured to process the scalar embeddings 46 via the vector scalar interactive message passing mechanism 44 of the message passing sub-block 40 of the MPGNN 24 to thereby generate and pass the scalar information from the scalar embeddings 46 to an embedding space containing the vector embeddings 48. The vector scalar interactive message passing mechanism 44 includes the scalar message function 44A and the vector message function 44B, and the embedding space is a latent space in memory accessible by both of these functions 44A, 44B.

Referring to FIG. 2, the one or more processors 12 are further configured to update, via a vector embedding update function 52 of the update sub-block 42, the vector embeddings 48 based on the embedding space containing the scalar information from the scalar embeddings 46 and the vector embeddings 48, to thereby produce updated vector embeddings 48A.

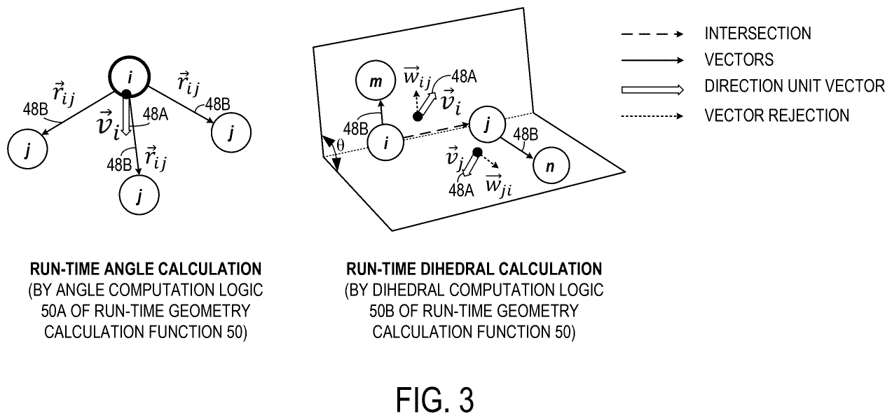

The one or more processors 12 are further configured to update, via a scalar edge embedding update function 54 and scalar node embedding update function 56 of the update sub-block 42, the scalar embeddings 46 (including scalar target node embeddings 46C and scalar edge embeddings 46A) based on run-time geometry calculations of the geometric relationships encoded in the vector embeddings 48 (and more specifically based on the direction unit vector 48A thereof), to thereby produce updated scalar embeddings (including updated scalar edge embedding 46A′ and updated scalar target node embedding 46C′) for the target node, with scalar source node embeddings 46B being updated on a separate pass through the update sub-block when the source node is the target node. The run-time geometry calculations are performed by a run-time geometry calculation function 50 of the update sub-block 42, further details of which are given below. The run-time geometry calculation function 50 includes angle computation logic 50A and dihedral computation logic 50B. As shown in FIG. 3, the angle computation logic 50A is configured to compute the direction unit vector 48A for the target node as well as each relational position bond angle vector 48B for edges connecting the target node to source nodes. It will be appreciated that the direction unit vector 48A is an equivariant vector representation of the change in position, and hence energy, of the target node i. Typically, these are computed only for source nodes within a threshold distance of a target node, i.e., within the neighborhood of the target node. Further as shown in FIG. 3, the dihedral computation logic 50B is configured to compute the dihedral angle θ between two edges based on a projection of the direction unit vectors 48A for each node i, j along the respective edges to nodes m and n.

Returning to FIG. 1, the one or more processors 12 are further configured to compute, via the update sub-block 42, an updated molecular graph 30A based on the updated scalar embeddings 46A′, 46C′ and updated vector embeddings 48′ for each node in the molecular graph 30A, by considering each node N independently as a target node.

Returning to FIG. 1, the one or more processors 12 are further configured to output, via the output block 36, a value for the target molecular property 38 of the molecular system 29 determined based on the updated molecular graph 30A.

As shown, the value for the target molecular property 38 along with the updated molecular graph 30A can be output to a downstream program 39 for further processing as well as being output to data storage 16 as inference results 28. As an example, the downstream program 39 may be a molecular dynamics simulation program for use in a molecular dynamics simulation 39A over multiple timesteps. For example, the potential energy of each node in the updated molecular graph 30A may be computed, and the potential energy of all nodes may be summed to calculate the total potential energy as the target molecular property 38. Similarly, interatomic forces may be computed along each of the edges based on the potential energies at each node, as described below.

The one or more processors 12 are configured to process the scalar embeddings 46 via the vector scalar interactive message passing mechanism 44 of the message passing sub-block 40 at least in part by, for each MPGNN processing block 34, for each of a plurality of target nodes in the molecular graph 30, in a MPGNN processing block loop: generate an edgewise scalar message 58 for each edge connected to the target node, via the scalar message function 44A of the message passing sub-block, pass the edgewise scalar message 58 to the vector message function 44B of the message passing sub-block 40, and generate an aggregated vector message 60, via the vector message function 44B, based on the vector embeddings 48 and the edgewise scalar message 58 from each of the edges connected to the target node, which are aggregated together for the target node through a vector scalar aggregation function 62. Thus, the one or more processors 12 may be configured to, in the MPGNN processing block loop prior to updating the vector embeddings and scalar embeddings, respectively aggregate the vector embeddings and scalar embeddings for each target node across the source nodes connected to the target node to generate a respective aggregated scalar message and aggregated vector message for the target node. The scalar message encoding information includes or is based on one or more of (and typically all of) the scalar node embeddings for the target node and neighbor (i.e., connected and within a threshold distance away) source nodes, the scalar edge embedding for the target node, and computed attention scores from a trained graph attention network for the one or more scalar node embeddings and scalar edge embedding for the target node.

The one or more processors are configured to generate the scalar message, at least in part by fusing the scalar node embeddings and scalar edge embeddings to thereby generate fused scalar embeddings. The fusing can be accomplished by concatenation, Hadamard product, or addition of a learnable bias term, or other suitable technique. Further, computing the attention scores can be based on the fused scalar embeddings via a non-linear activation function, or other suitable activation function.

In the MPGNN processing block loop, the one or more processors can be configured to update, via the update sub-block, the vector embeddings at least in part by updating, via the update sub-block, the vector embeddings for the target node based on the aggregated scalar messages and the aggregated vector messages for the target node.

In the MPGNN processing block loop, the one or more processors are configured to update, via the update sub-block, the scalar embeddings at least in part by performing, via the update sub-block, the run-time geometry calculations to compute run-time values for the relative position bond angle vectors, the direction unit vector, and a dihedral angle for each target node, computing, via the update sub-block, an updated scalar node embedding for the target node based on the computed relative position bond angle vector, the aggregated scalar messages for the target node, and the scalar node embedding for the target node, and computing, via the update sub-block, an updated scalar edge embedding based on the computed dihedral angle and the scalar edge embedding. The one or more processors can be configured to compute the updated molecular graph based on the updated scalar edge embedding, the updated scalar node embedding, and the updated vector embedding.

The following paragraphs provide additional description of implementation details of a particular embodiment of the MPGNN 24, referred to as Vector Scalar interactive Graph Neural Network (ViSNet), which can be implemented by the one or more processors 12 of computing system 10 described above. In the following paragraphs, reference is generally made to FIGS. 1-2 with sparing use of reference characters to avoid confusion with mathematical symbols for the embeddings, vectors, functions, and output parameters thereof. References to ViSNet hereinbelow should be understood as referring to a particular implementation of MPGNN 24, and similar terms are used to describe similar components as described above. At a high level, ViSNet embeds the 3D structures of molecules and extracts the geometric information through a series of ViSNet blocks and outputs the energy and forces through an output block. The energy and forces predicted by ViSNet can be used to drive molecular dynamics simulations. Referring to FIG. 2, as briefly discussed above, a ViSNet block consists of two sub-blocks: a message sub-block and an update sub-block. These two sub-blocks enact a Vector Scalar interactive Message Passing mechanism, one implementation of which is referred to herein as ViS-MP) that implements equations Eq. 5 to Eq. 9. Concretely, in the message sub-block, the scalar messages mᵢⱼ and vector messages \vec{m}ᵢⱼ are first obtained through message function ϕₘ from node embedding hᵢ, edge embedding fᵢⱼ for each edge connecting a target node i to a source node j, relative position vectors \vec{r}ᵢⱼ (also referred to as relative position bond angle vectors, which are vectors between the target node i and each source node j), and direction unit vector \vec{v}ᵢ. Then, the scalar messages mᵢⱼ and vector messages \vec{m}ᵢⱼ are aggregated to the target node i. In the update sub-block, hᵢ is updated by the aggregated scalar message mᵢ and the output of angle computation logic of the run-time geometry calculation function from \vec{v}ᵢ through an update function

$${ϕ}_{u n}^{s} .$$

Then fᵢⱼ is updated by the output of dihedral computation logic of the run-time geometry calculation function from \vec{v}ᵢ and \vec{v}ⱼ through an update function

$${ϕ}_{u e}^{s} .$$

FIG. 3 shows the angles computed by the run-time geometry calculation function. Returning to FIG. 2, finally, the vector embedding \vec{v}ᵢ is updated by both scalar and vector messages through an update function

$${ϕ}_{u n}^{v} .$$

The overall design of VIS-MP aims to improve the interaction between scalar and vector embeddings. FIG. 3 shows an illustration of directional geometric information that is extracted by the RGC function with linear complexity. The RGC function includes two sets of programming logic—angle computation logic and dihedral computation logic. In the RGC function, the equivariant vector representation (termed as “direction unit vector” herein) for each node is designed to preserve geometric information for the node. Through rejections and inner products between two direction unit vectors for nodes i and j in FIG. 3 (See Eq. 1-Eq. 4), the angle and dihedral information can be directly obtained with lower computational complexity of O(N) for both angle and dihedral calculation.

ViSNet is a versatile GDL model for predicting energy potentials which can predict potential energy, atomic forces, as well as various quantum chemical properties by taking atomic coordinates and atomic numbers as inputs. As shown in FIG. 1, the ViSNet model is composed of an embedding block and multiple stacked ViSNet blocks, followed by an output block. The atomic number and coordinates are fed into the embedding block followed by ViSNet blocks to extract and encode geometric representations. The geometric representations are then used to predict molecular properties through the output block. It is worth noting that ViSNet is an energy-conserving potential model, i.e., the predicted atomic forces are derived from the negative gradients of the potential energy with respect to the atomic coordinates.

As shown in FIG. 2 and briefly discussed above, each ViSNet block consists of a message sub-block and an update sub-block. These blocks work together as parts of a vector scalar interactive message passing mechanism, referred to as ViS-MP. The rich geometric information embedded in messages passed via ViS-MP is extracted by the RGC function with linear complexity. The RGC function and ViS-MP will be explained in detail in the following paragraphs.

Turning now to the RGC function, the success of classical force fields shows that geometric features such as interatomic distances, angles, and dihedrals are useful to determine the total potential energy of molecules. The explicit extraction of invariant geometric representations in prior approaches often suffer from the drawbacks of large amounts of time or memory being consumed during model training and inference. Given an atom, the calculation of angular information scales as order N squared O(N²) with the number of neighboring atoms, while the computational complexity is order N cubed O(N³) for dihedrals. To alleviate this problem, a RGC function is proposed that uses an equivariant vector representation referred to as a direction unit vector for each node to preserve its geometric information. RGC directly calculates the geometric information from the direction unit vector, which only sums the vectors from the target node i to its neighbor source nodes j once. Therefore, the computational complexity can be reduced to O(N).

Considering the sub-structure of an example molecule with four atoms shown in FIG. 3, the angular information of the target node i could be obtained from the vector \vec{r}ᵢⱼ as follows:

$${\vec{u}}_{ij} = \frac{{\vec{r}}_{i j}}{‖ {\vec{r}}_{i j} ‖} , \qquad ( 1 )$$

$${\vec{v}}_{i} = {\sum }_{j = 1}^{{N}_{i}} {\vec{u}}_{ij}$$

$${‖ {\vec{v}}_{i} ‖}^{2} = {\sum }_{j = 1}^{{N}_{i}} {\sum }_{k = 1}^{{N}_{i}} 〈 {\vec{u}}_{ij} , {\vec{u}}_{ik} 〉 = {\sum }_{j = 1}^{{N}_{i}} {\sum }_{k = 1}^{{N}_{i}} cos {θ}_{jik} \qquad ( 2 )$$

where \vec{r}ᵢⱼ is the vector from node i to its neighboring source node j, \vec{u}ᵢⱼ is the unit vector of \vec{r}ᵢⱼ. Here, the direction unit vector \vec{v}ᵢ of node i is proposed as the sum of all unit vectors from node i to its all neighboring nodes j, where node i is the intersection of all unit vectors. As shown in Eq. 2, the inner product of direction unit \vec{v}ᵢ of node i which represents the sum of inner products of unit vectors from node i to all its neighboring nodes, is calculated. Combining with Eq. 1, the inner product of direction \vec{v}ᵢ finally stands for the sum of cosine values of all angles formed by node i and any two of its neighboring nodes.

Similar to runtime angle calculation, the vector rejection of the direction unit \vec{v}ᵢ of node i and \vec{v}ⱼ of node j on the vector \vec{u}ᵢⱼ and \vec{u}ⱼᵢ respectively is calculated.

$${\vec{w}}_{i j} = {Rej}_{{\vec{u}}_{ij}} ( {\vec{v}}_{i} ) = {\vec{v}}_{i} - 〈 {\vec{v}}_{i} , {\vec{u}}_{ij} 〉 \cdot {\vec{u}}_{ij} = {\sum }_{m = 1}^{{N}_{i}} {Rej}_{{\vec{u}}_{ij}} ( {\vec{u}}_{im} ) \qquad ( 3 A )$$

$${\vec{w}}_{ji} = {Rej}_{{\vec{u}}_{ji}} ( {\vec{v}}_{j} ) = {\vec{v}}_{j} - 〈 {\vec{v}}_{j} , {\vec{u}}_{ji} 〉 \cdot {\vec{u}}_{ji} = {\sum }_{n = 1}^{{N}_{j}} {Rej}_{{\vec{u}}_{ji}} ( {\vec{u}}_{jn} ) \qquad ( 3 B )$$

where Rej_{\vec{b}}(\vec{a}) represents the vector component of \vec{a} perpendicular to \vec{b}, termed as the vector rejection. \vec{u}ᵢⱼ and \vec{v}ᵢ are defined in Eq. 1. \vec{w}ᵢⱼ represents the sum of the vector rejection Rej_{\vec{u}}_{ᵢⱼ}(\vec{u}ᵢₘ) and \vec{w}ⱼᵢ represents the sum of the vector rejection Rej_{\vec{u}}_{ⱼᵢ}(\vec{u}ⱼₙ). The inner product between \vec{w}ᵢ and \vec{w}ⱼ is then calculated to conduct dihedral information of the axis of \vec{u}ᵢⱼ as follows:

$$〈 {\vec{w}}_{ij} , {\vec{w}}_{j i} 〉 = {\sum }_{m = 1}^{{N}_{i}} {\sum }_{n = 1}^{{N}_{j}} 〈 {Rej}_{{\vec{u}}_{ij}} ( {\vec{u}}_{im} ) , {Rej}_{{\vec{u}}_{ji}} ( {\vec{u}}_{ji} ) 〉 = {\sum }_{m = 1}^{{N}_{i}} {\sum }_{n = 1}^{{N}_{j}} cos {φ}_{mijn} \qquad ( 4 )$$

By calculating the inner product of the vector \vec{w}ᵢⱼ with the vector \vec{w}ⱼᵢ, the sum of cosine values of all dihedrals ₘᵢⱼₙ with eᵢⱼ may be obtained as the common rotation axis as shown in FIG. 3. Note that the directional unit vector is not restricted to Cartesian coordinates but can be extended to higher order tensors by spherical harmonics.

Turning now to the details of ViS-MP, in order to make effective use of geometric information and to enhance the interaction between scalars and vectors, a vector scalar interactive message passing mechanism (ViS-MP) with respect to the intersecting nodes and edges for angles and dihedrals respectively is designed. The following operations are performed by ViS-MP:

$${m}_{i}^{l} = {\sum }_{j ∈ N ( i )} {ϕ}_{m}^{s} ( {h}_{i}^{l} , {h}_{j}^{l} , {f}_{i j}^{l} ) \qquad ( 5 )$$

$${\vec{m}}_{i}^{l} = {\sum }_{j ∈ N ( i )} {ϕ}_{m}^{v} ( {m}_{ij}^{l} , {\vec{r}}_{ij} , {\vec{v}}_{j}^{l} ) \qquad ( 6 )$$

$${h}_{i}^{l + 1} = {ϕ}_{u n}^{s} ( {h}_{i}^{l} , {m}_{i}^{l} , 〈 {\vec{v}}_{i}^{l} , {\vec{v}}_{i}^{l} 〉 ) \qquad ( 7 )$$

$${f}_{ij}^{l + 1} = {ϕ}_{ue}^{s} ( {f}_{ij}^{l} , 〈 {Rej}_{{\vec{r}}_{ij}} ( {\vec{v}}_{i}^{l} ) , {Rej}_{{\vec{r}}_{ji}} ( {\vec{v}}_{j}^{l} ) 〉 ) \qquad ( 8 )$$

$${\vec{v}}_{i}^{l + 1} = {ϕ}_{u n}^{v} ( {\vec{v}}_{i}^{l} , {m}_{i}^{l} , {\vec{m}}_{i}^{l} ) \qquad ( 9 )$$

where hᵢ denotes the scalar embedding of node i, fᵢⱼ stands for the edge feature between node i and node j. \vec{v}ᵢ represents the embedding of direction unit vector mentioned in the description above of the RGC function. The superscript of variables indicates the index of the block to which the variables belong. ViS-MP extends the conventional message passing, aggregation, and update processes with vector-scalar interactions. Eq. 5 and Eq. 6 depict the message passing and aggregation processes. To be concrete, scalar messages mᵢⱼ incorporating scalar embedding hⱼ, hᵢ, and fᵢⱼ are passed and then aggregated to node i through a message function

$${ϕ}_{m}^{s}$$

(Eq. 5). Similar operations are applied for vector messages

$${\vec{m}}_{i}^{l}$$

or node i that incorporates scalar message mᵢⱼ, vector \vec{r}ᵢⱼ and vector embedding \vec{v}ⱼ (Eq. 6). Eq. 7 and Eq. 8 demonstrate the update processes. As shown, hᵢ is updated by the aggregated scalar message output mᵢ while the inner product of \vec{v}ᵢ is updated through an update function

$${ϕ}_{u n}^{s} .$$

Then \vec{f}ᵢⱼ is updated by the inner product of the rejection of the vector embedding \vec{v}ᵢ and \vec{v}ⱼ through an update function

$${ϕ}_{u e}^{s} .$$

Finally, the vector embedding \vec{v}ᵢ is updated by both scalar and vector messages through an update function

$${ϕ}_{u n}^{v} .$$

Notably, the non-linear functions for vectors, i.e., ϕ^{v} are equivariant. Details regarding the message and update functions can be found below.

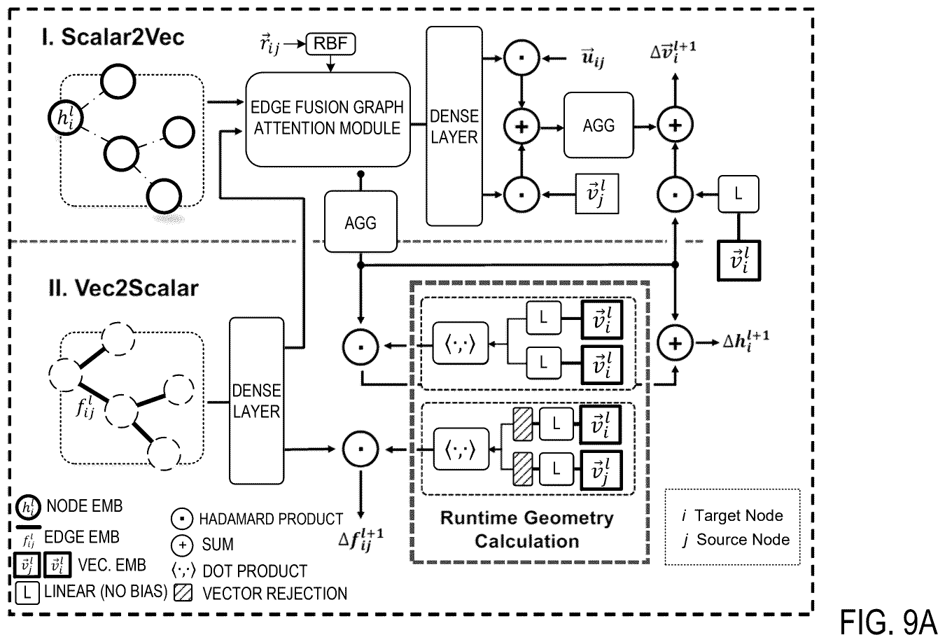

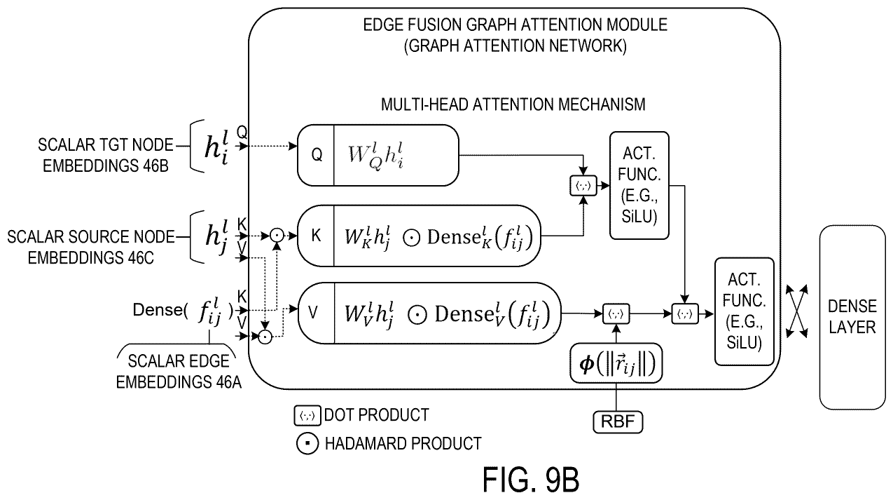

Referring now to FIG. 9A, a more complete description of the operation of ViS-MP will now be explained. FIG. 9A shows the general workflow of scalar and vector embeddings. Specifically, one ViSNet block consists of two modules: i) Scalar2Vec, which attaches scalar embeddings to vectors; ii) Vec2Scalar, which renovates scalar embeddings built by the RGC function. The inputs of Scalar2Vec are the node embedding hᵢ, edge embedding fᵢⱼ, direction unit vector \vec{v}ᵢ and the relative positions. The edge fusion graph attention module (which functions as

$${ϕ}_{m}^{s} )$$

takes as input hᵢ and the output of the dense layer following fᵢⱼ, and outputs scalar messages. Before aggregation, each scalar message passes through a dense layer, and is fused with the relative position unit vector \vec{u}ᵢⱼ and its own direction unit vector \vec{v}ⱼ. A dense layer is a layer that is deeply (fully) connected with its preceding layer (i.e., the output of the edge fusion graph attention module), and it functions to change the dimension of the output of the preceding layer by performing matrix vector multiplication. Then, the vector messages are computed and the computed vector messages among the neighborhood are aggregated. Through a gated residual connection, the final residual Δ\vec{v}ᵢ is produced. In Vec2Scalar module, the final Δhᵢ is computed by taking the Hadamard product of the aggregated scalar message and the output of the angle computation logic of the RGC function and adding a gated residual connection. Likewise, the final Δfᵢⱼ is determined by combining the projected fᵢⱼ and the output of dihedral computation logic of the RGC function.

In summary, the geometric features are extracted by taking the inner products with the RGC function outputs and the scalar and vector embeddings are cyclically updating each other in ViS-MP so as to learn a comprehensive geometric representation from the molecular graph.

ViSNet can be used to make accurate quantum chemical property predictions. As evidence of this, ViSNet has been evaluated on several prevailing benchmark datasets including MD17, revised MD17 (termed as “rMD17”), and QM9 for energy, force, and other molecular property predictions. MD17 consists of the MD trajectories of 7 small organic molecules, and the number of conformations in each molecule dataset ranges from 133,700 to 993,237. The dataset rMD17 is a reproduced version of MD17 with higher accuracy. QM9 consists of 12 kinds of quantum chemical properties of 133,385 small organic molecules with up to 9 heavy atoms. ViSNet was compared with the results of other state-of-the-art algorithms for molecular property prediction, including the kernel-based algorithms FCHL 19 and GAP, the directional information-based algorithms SchNet, ANI, PhysNet, EGNN, ACE, DimeNet/DimeNet++, GemNet, PaiNN, and ET, and the group representation theory-based algorithms UNITE and NequIP. The training details of ViSNet on each benchmark are described below.

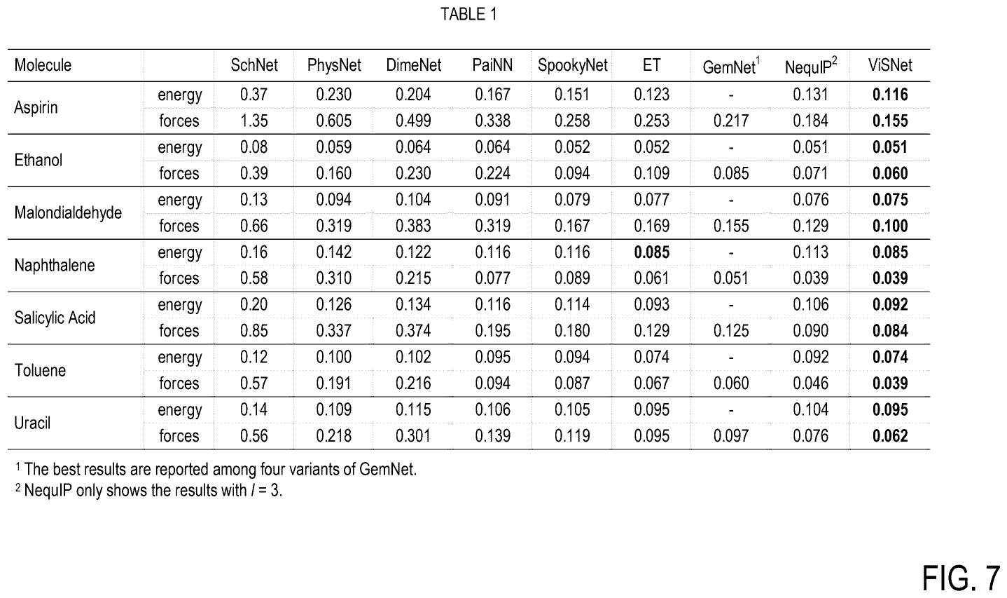

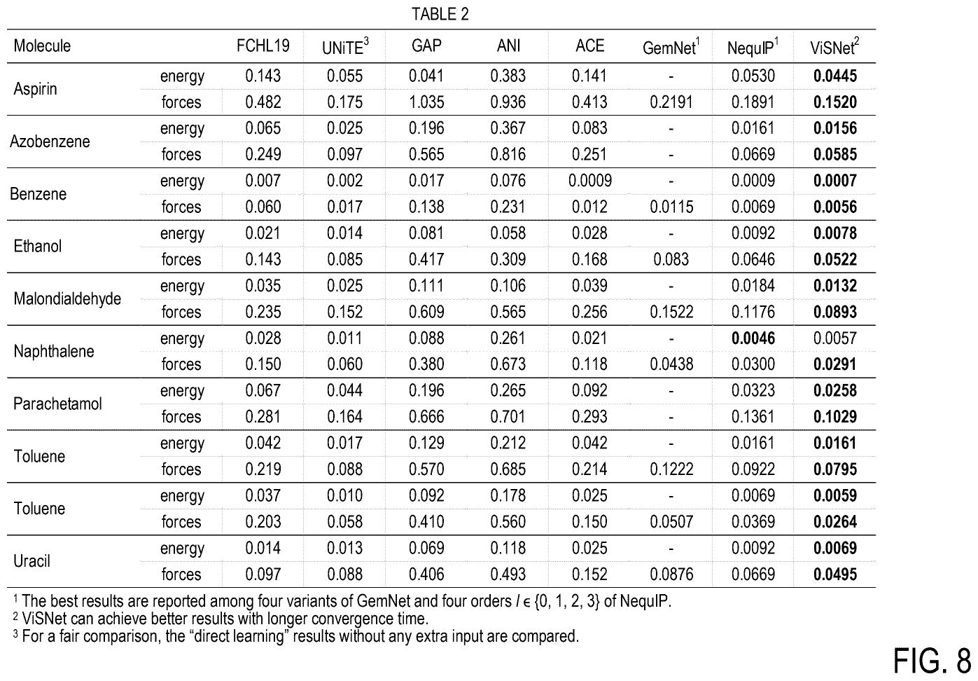

As shown in Table 1 of FIG. 7 and Table 2 of FIG. 8, it is remarkable that ViSNet outperformed the algorithms to which it was compared for all molecules with lowest mean absolute errors (MAEs) for both predicted energies and forces. Table 1 shows mean absolute errors (MAE) of energy (kcal/mol) and force (kcal/mol/° A) for 7 small organic molecules on MD17 compared with state-of-the-art algorithms. The best result in each category is highlighted in bold. Table 2 shows mean absolute errors (MAE) of energy (kcal/mol) and force (kcal/mol/° A) for 10 small organic molecules on rMD17 compared with compared with state-of-the-art algorithms. The best result in each category is highlighted in bold. Although there only 950 samples of each kind of molecule were used to train ViSNet and another 50 samples were used in the validation set, ViSNet still outperformed the kernel-based algorithms by a large margin, which indicates the equivariant model design in ViSNet captures geometric information efficiently and thus significantly alleviates the requirements of a large number of training samples. On the one hand, compared with PaiNN and ET, ViSNet incorporates more directional geometric information through the strategies of the RGC function, which appears to contribute to performance gains. On the other hand, given that angle and dihedral information are adopted in Gem-Net, the superior performance of ViSNet to Gem-Net indicates ViS-MP can better leverage geometric information during message passing to achieve improved results.

Furthermore, ViSNet also achieved superior performance for quantum chemical property predictions on QM9. Extended Data Table 1 of FIG. 12 shows Mean Absolute Errors (MAE) of 12 kinds of molecular properties on QM9 compared with state-of-the-art algorithms. The best result in each category is highlighted in bold. As shown, ViSNet outperformed the algorithms to which it was compared for 11 of 12 chemical properties and achieved the comparable result on the remaining property.

Molecular dynamics simulation is one useful application of the predicted potential energy and atomic forces from ViSNet. To evaluate ViSNet as the potential energy prediction model for ab initio molecular dynamics simulations, an instance of ViSNet was created that was trained only with 0.7% of samples available (i.e., 950 samples for model training) on MD17 in the ASE simulation framework, to perform ab initio MD simulations for all 7 kinds of organic molecules. In this analysis, all simulations were run with a time step τ=0.5 fs under Berendsen thermostat with the other settings the same as those of the MD17 dataset.

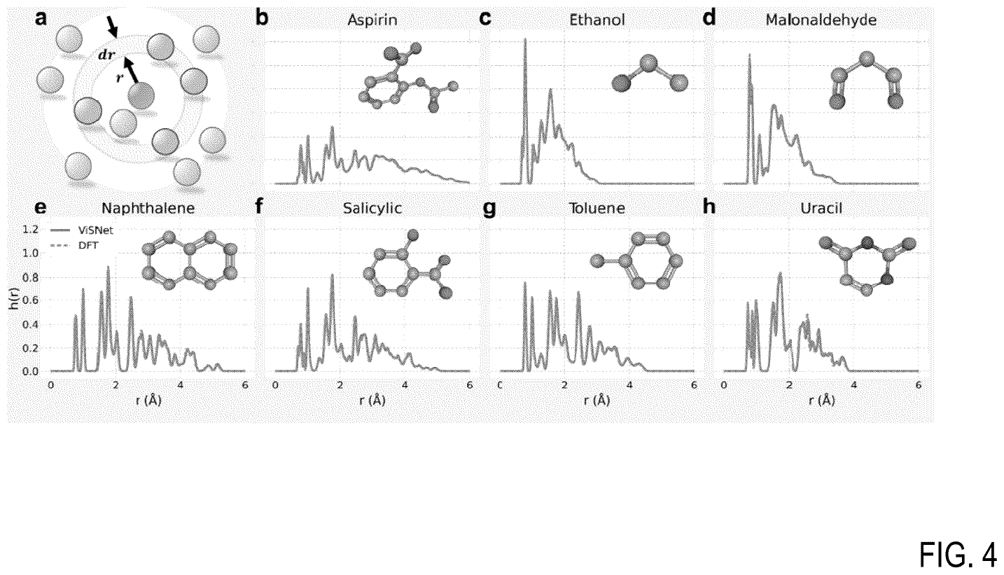

FIG. 4 shows the interatomic distance distributions of molecular dynamics simulations driven by ViSNet and DFT. At (a), an illustration is shown of the atomic density at a radius r with an arbitrary atom as the center. The interatomic distance distribution h(r) is defined as the ensemble average of atomic density at a radius r. FIG. 2 at (b-h) illustrates that the distributions derived from ViSNet are very close to those generated by DFT. The interatomic distance distribution is defined as the ensemble average of atomic density. At (b) to (h) interatomic distance distribution comparisons between simulations by ViSNet and DFT for all seven organic molecules in MD17 are shown. The interatomic distance distributions shown were derived from AIMD simulations with ViSNet as the potential energy model, and with ab initio molecular dynamics simulations at the DFT level, for all 7 molecules respectively. Results of ViSNet are shown using a solid line, while a dashed line is used for the DFT results. As can be seen, the curves resulting from the DFT and ViSNet results are almost indistinguishable. The structures of the corresponding molecules are shown in the upper right corner on each panel (b)-(h) in FIG. 4.

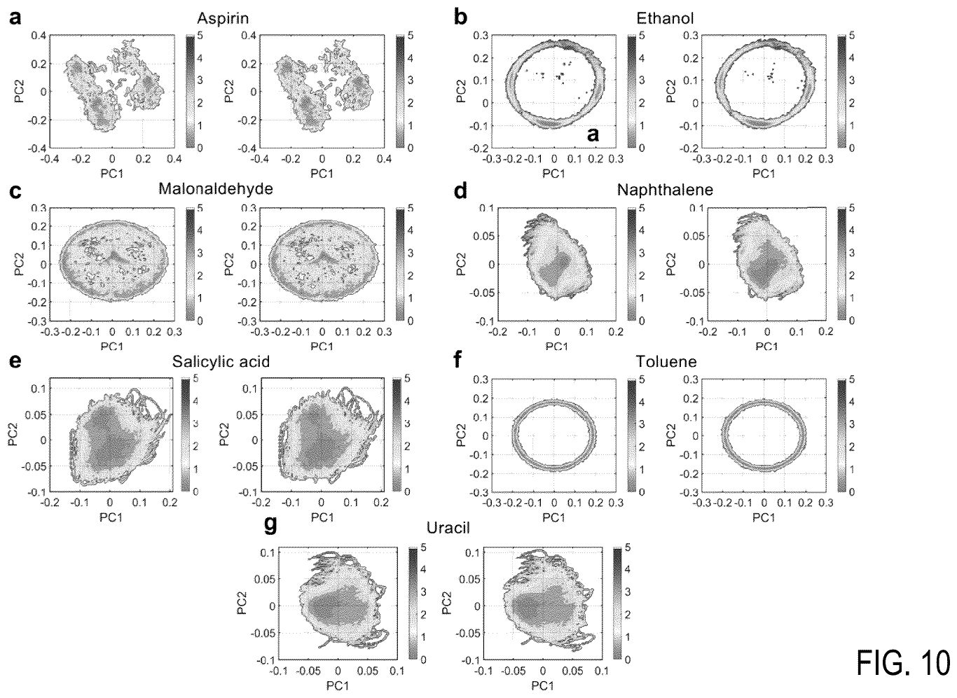

The potential energy surfaces sampled by ViSNet and DFT for these molecules respectively are compared in the FIG. 10. FIG. 10 illustrates potential energy surfaces (PES) explored by a molecular dynamics simulation driven by ViSNet and DFT for the 7 molecules in MD17. Potential energy surfaces were plotted to allow inspection of the conformational ensemble from simulations driven by DFT and ViSNet. The snapshots in each simulation trajectory were aligned to the initial structure of the corresponding molecule in MD17. Then, principal component analysis (PCA) was applied on the coordinates for each conformation. PC1 and PC2 have been set as two axes. The potential energy values were calculated as ΔG(x, y)=k_{B}Tlng(x, y), where k_{B} is the Boltzmann constant, T is the temperature of systems and g(x, y) represents the normalized joint probability distribution. The minimum energy value was set to zero. 100 bins were applied to generate the landscape in both x and y axis. In each panel, the left and right subfigures show the PES of the same molecule obtained by DFT and ViSNet, respectively.

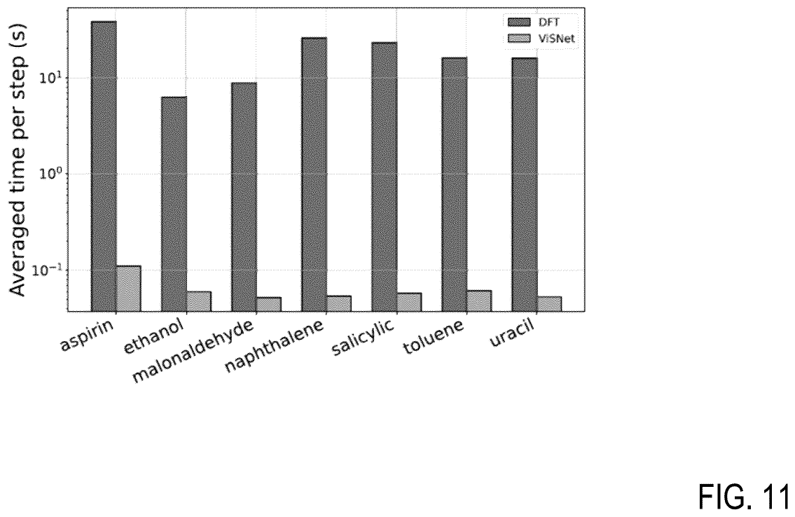

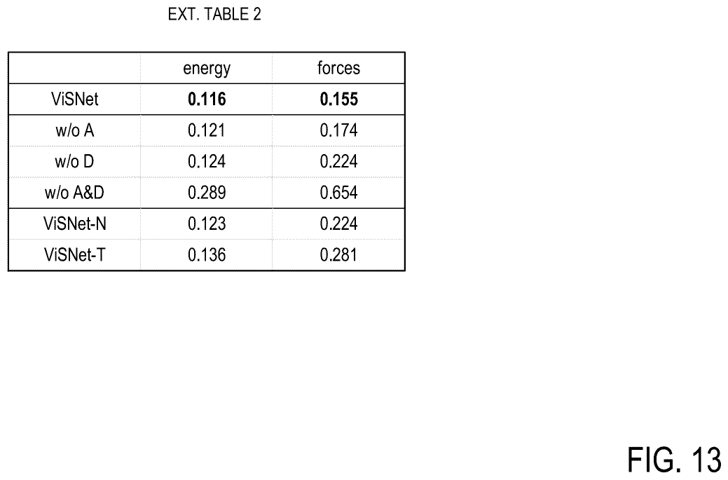

The consistent potential energy surfaces shown in FIG. 10 suggest that ViSNet can recover the kinetic properties and the conformational space from the simulation trajectories well, indicating the usefulness of ViSNet for real molecular dynamics simulation. Furthermore, compared with the prohibitive computational cost of DFT, ViSNet dramatically saves computational time by 2-3 orders of magnitude, as shown in FIG. 11 and Extended Table 2 of FIG. 13. These results demonstrate that with only a few of training samples, ViSNet can act as the potential energy prediction model to perform high-fidelity molecular dynamics simulations with much less computational cost.

FIG. 11 illustrates the time taken to perform molecular dynamics simulations driven by ViSNet and DFT. Simulations were run with the ASE framework for the 7 molecules in MD17. The average time taken for each simulation step is recorded in seconds. The y axis is a logarithmic axis with a base of 10. As shown ViSNet computations have a significantly lower average time per step than DFT calculations.

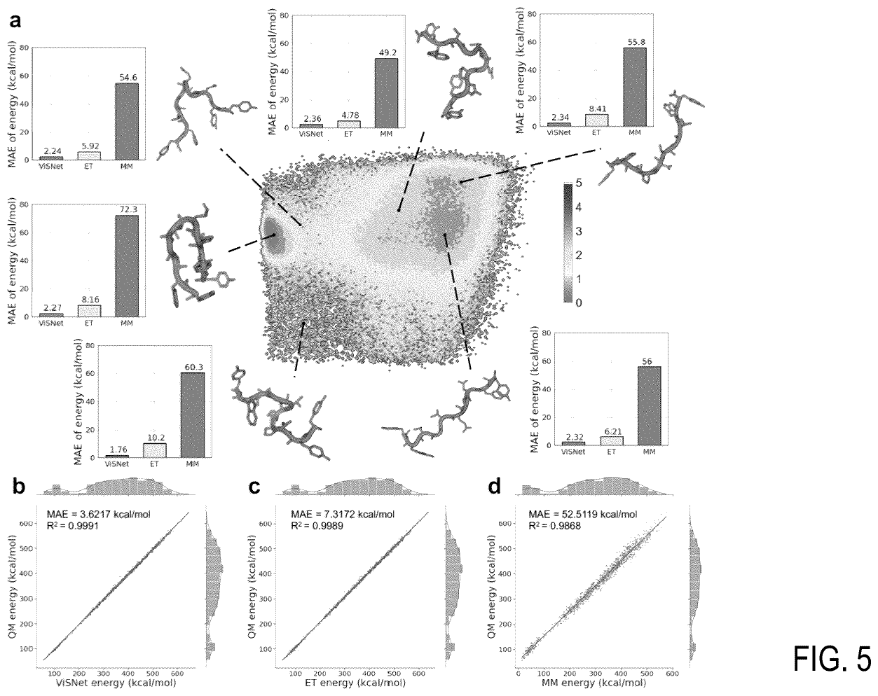

FIG. 5 shows a visualization of the energy landscape of Chignolin and evaluations of energy prediction by ViSNet, Equivariant Transformations (ET), and Molecular Mechanics (MM) approaches. At (a), the energy landscape of Chignolin sampled by REMD is shown. The x-axis of the landscape is the distance between mainchain O on Y2 and mainchain N on G6, while the y-axis is the distance between mainchain O on E4 and mainchain N on T7. Six representative structures are then selected for visualization. Each structure is shown as cartoon and residues are depicted in sticks. The histograms show the mean absolute error (MAE) between the energy difference predicted/calculated by ViSNet, ET, MM, and the ground truth calculated by DFT on the corresponding structure. At (b) to (d) the energy correlations on the test dataset between the ground truth calculated by DFT and the predictions made by ViSNet, ET, and molecular mechanics are shown. The corresponding distributions of energy predictions or calculations as well as the ground truth are shown in each panel.

ViSNet can be applied to real-world proteins to explore its scalability from small organic molecules to large biomolecules. Considering that the time complexity of DFT roughly scales on an order of N cubed O(N³) with the number of atoms, the simplest protein Chignolin with 166 atoms is employed to build an MD dataset at DFT level for model training and evaluation. For data generation, an 80 ns Replica Exchange Molecular Dynamics (REMD) simulation was run to sample various folding and unfolding states of Chignolin. As a result, 9,543 representative conformations were collected and the energy and forces on nuclei were calculated by a Gaussian 16 software package. It is believed that this is the first MD dataset for real-world full-atom proteins at the DFT level. The data generation process is elaborated in below. The Chignolin dataset was split into training, validation, and test sets by the ratio of 8:1:1. ViSNet, as well as models to which it is compared, were trained with the best performance in the evaluations elaborated below including ET, NequIP, and GemNet on the Chignolin dataset with their default settings on Tesla V100 GPUS. During model training, GemNet failed due to running out of GPU memory even though the batch size was set to 1, while NequIP suffered from under-fitting with its default hyperparameters on the Chignolin dataset. ViSNet and ET could successfully be trained and compared with molecular mechanics (MM). DFT results were used as the ground truth. FIG. 5 at (a) shows the free energy landscape of Chignolin sampled by REMD and depicted by d_{Y2-G6} (the distance between mainchain O on Y2 and main-chain Non G6) and d_{E4-T7} (the distance between mainchain O on E4 and mainchain Non T7). The concentrated energy basin on the left shows the folded state and the scattered energy basin on the right shows unfolded state. Six representative structures are picked in the low potential energy regions with both folded and unfolded states and some intermediate states were selected with high potential energy. The energy predictions for the six representative structures were visualized, and ViSNet produced a significantly better estimation of the potential energy than either of ET trained on the same dataset and MM with empirical force fields. FIG. 5 at (b) to (d) shows the correlations between the predicted energies by ViSNet, ET, MM, and the ground truth values given by DFT for the conformations in the test set. ViSNet achieved the lowest MAE and the highest R2 score. These results suggest that ViSNet has the ability to scale to real-world proteins with a small training set and achieve superior accuracy and efficiency.

To further explore where the performance gains of ViSNet come from, a comprehensive ablation study was conducted. Specifically, the run-time angle calculation logic (w/o A), runtime dihedral calculation logic (w/o D), and both of these (w/o A&D) were excluded in an ablation study of ViSNet performance, in order to evaluate the usefulness of each part. Further some model variants were designed with different message passing mechanisms based on ViS-MP for scalar and vector interaction. For example, ViSNet-N was designed to directly aggregate the dihedral information to intersecting nodes, and ViSNet-T was designed to leverage another form of dihedral calculation. The results of the ablation study are shown in Extended Table 2 in FIG. 13. Specifically, Extended Table 2 shows the results of the ablation study of ViSNet on aspirin in the MD17 dataset. Extended Table 2 lists the results of ViSNet and its variants without runtime angle calculation (w/o A), without runtime dihedral calculation (w/o D), and without either of them (w/o A&D), and as compared to message passing variants ViSNet-N and ViSNet-T. The best results are shown in bold. Based on the results, it can be concluded that both kinds of directional geometric information are useful, and the dihedral information contributes some to the final performance. Furthermore, the significant performance drop from ViSNet-N and ViSNet-T further validate the effectiveness of ViS-MP mechanism.

The visualization and interpretability of ViSNet on molecular structures will now be discussed. To explain how incorporating geometric features such as angles and dihedrals improves the expressiveness of ViSNet, a model of interpretability of ViSNet is illustrated by mapping the angle and dihedral representations derived from inner product of direction unit vectors in the model to the atoms and bonds of the molecular structure. The gap between geometric representation in ViSNet and molecular structures may be bridged. The embeddings are visualized after the inner product of direction unit vectors [symbol] \vec{v}ᵢ, \vec{v}ᵢ [symbol] and [symbol] \vec{w}ᵢⱼ, \vec{w}ⱼᵢ [symbol] extracted from 50 aspirin samples on the validation set. The high-dimensional embeddings are reduced to 2-dimensional space using T-SNE and then clustered using DBSCAN without the prior of the number of clusters.

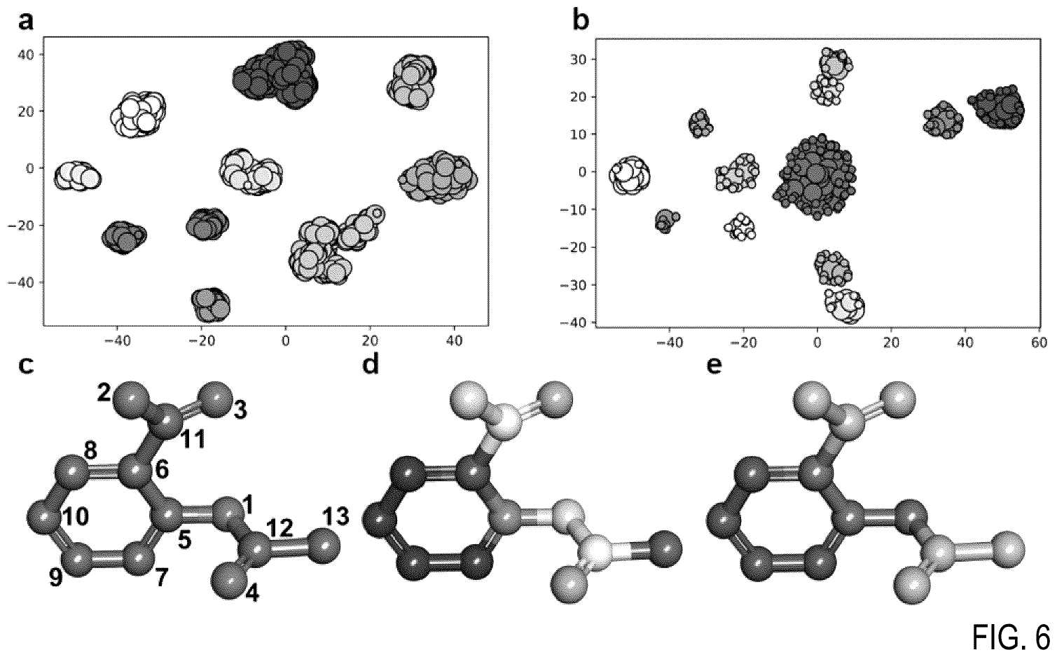

FIG. 6 at (a) and (b) shows the clustering results of embeddings after taking the inner product of the direction unit vectors. FIG. 6 at (d) and © maps back the clustering results on aspirin chemical structure while FIG. 6 at (c) shows the atoms of aspirin labeled with indices. Interestingly, the embeddings for both intersecting node and edge could be distinctly gathered into several clusters shown in different colors. For example, although carbon atom C₁₁ and carbon atom C₁₂ possess different positions and connect with different atoms, their inner products [symbol] \vec{v}ᵢ, \vec{v}ᵢ [symbol] are clustered into the same class for holding similar substructures ({C₋₁—O₂O₃C₆} and {C₋₂—O₁O₄C₁₃}).

FIG. 6 is a visualization to evaluate the model interpretability of ViSNet. FIG. 6 at (a) shows clusters of embeddings after [symbol] \vec{v}₁, \vec{v}ᵢ [symbol] . The [symbol] \vec{v}ᵢ, \vec{v}ᵢ [symbol] represents angle representations with the intersecting node i as the vertex. FIG. 6 at (b) shows clusters of embeddings after [symbol] \vec{w}ᵢⱼ, \vec{w}ⱼᵢ [symbol] . The [symbol] \vec{w}ᵢⱼ, \vec{w}ⱼᵢ [symbol] represents dihedral representations with the intersecting edge eᵢⱼ as the common rotation axis. FIG. 6 at (c) shows the chemical structure of aspirin. Carbon and oxygen atoms are shown in darker shading. FIG. 6 at (d) shows the chemical structure of aspirin, with atoms shaded according to the clusters in panel (a). FIG. 6 at (e) shows the chemical structure of aspirin with bonds shaded according to the clusters in panel (b). The hydrogen atoms in the chemical structure of aspirin in panel (c) to (e) are omitted for simplification.

For mapping [symbol] \vec{w}ᵢ, \vec{w}ⱼ [symbol] to edges in the aspirin chemical structure, more clusters are detected since distances of two atoms within a threshold are all taken into consideration. Here only the largest two clusters are illustrated in the panel shown in FIG. 6 at (b), which correspond to the aromatic bond and the C—O bond in FIG. 4(e). It is intuitive that the intersecting edges of {C₈, C₁₀}, {C₁₀, C₉}, and {C₉, C₇} possess the similar structures ({C₆—(C₈-C₁₀)—C₉}, {C₈—(C₁₀-C₉)—C₇}, and {C₁₀—(C₉-C₇)—C₅}). In addition, even if the intersecting edges of {O₂, C₁₁} and {O₃, C₁₁} belong to different types of chemical bonds, the intersecting edges are still classified into the same class since they possess similar structures ({(O₂—C₁₁)—C₆O₃} and {(O₃—C₁₁)—C₆O₂}). Thus, it can be concluded from this visualization that ViSNet can discriminate different molecular substructures in the embedding space.

Turning now to the detailed operation of ViSNet, ViSNet predicts the molecular properties (e.g., energy Ê, forces \vec{F}∈ [symbol] , dipole moment μ) from the current states of atoms, including the atomic positions X∈ [symbol] and atomic numbers Z∈ [symbol] . The architecture of the proposed ViSNet is shown in FIGS. 1-3. The overall design of ViSNet follows the vector scalar interactive message passing as illustrated from Eq. 5-Eq. 8. First, an embedding block encodes the atom numbers and edge distances into the embedding space. Then, a series of ViSNet blocks update the nodewise scalar and vector representations based on their interactions. A residual connection is placed between two ViSNet blocks. Finally, stacked corresponding gated equivariant blocks are attached to the output block for specific target molecular property prediction.

In the embedding block, ViSNet expands the direct node and edge embedding with their neighbors. It first embeds atomic chemical symbol zi, and calculates an edge representation for edges with distances within the cutoff through radial basis functions (RBF). The RBF, it will be appreciated, cuts off edges that are beyond a threshold distance, typically expressed in angstrom, for computational efficiency purposes. Then, the initial embedding of the atom i, its 1-hop neighbors j and the directly connected edge et within cutoff are fused together as the initial node embedding

$${h}_{i}^{0}$$

and edge embed-ding

$${f}_{ij}^{0} .$$

In summary, the embedding block is given by:

$${h}_{i}^{0} , {f}_{ij}^{0} = Embedding Block ( {z}_{i} , {z}_{j} , {e}_{ij} ) , j ∈ N ( i ) \qquad ( 10 )$$

N(i) denotes the set of 1-hop neighboring nodes of node i, and j is one of its neighbors. The initial vector embedding \vec{v}ᵢ is set to \vec{0}. The vector embeddings \vec{v} are projected into the embedding space; \vec{v}∈ [symbol] and F is the size of hidden dimension. The advantage of such projection is to assign a unique high-dimensional representation for each embedding to discriminate from each other. Further discussions on its effectiveness and interpretability are given in the Results section.

Referring to FIG. 9A, in the Scalar2Vec module, the vector embedding \vec{v} is updated by both the scalar messages derived from node and edge scalar embeddings (Eq. 5) and the vector messages with inherent geometric information (Eq. 6). The message of each atom is calculated through an Edge Fusion Graph Attention module, which fuses the node and edge embeddings and computes the attention scores. The fusion of the node and edge embeddings could be the concatenation operation, Hadamard product, or adding a learnable bias. A graph attention network with a multi-head head attention mechanism can be used to implement the edge fusion, with the Hadamard product.

Turning briefly to FIG. 9B, a detail view of the edge fusion graph attention module of FIG. 9A is shown. The edge fusion graph attention module implements a graph attention network, which combines the features of a graph neural network and one or more attention layers. As shown, the edge fusion graph attention module includes a multi-head attention mechanism that takes as input the scalar target node embeddings 46B (as Q values of the attention mechanism), the scalar source node embeddings 46C (as both K and V values of the attention mechanism), and scalar edge embeddings 46A (as both K and V values of the attention mechanism), along with the output of the RBF, which is the cosine cutoff of the scalar value of the magnitude of the radial vector along the edge ij. The fused representations are passed through a nonlinear activation function such as a Sigmoid Liner Unit (SiLU) function as shown in Eq. 11. The value (V) in the attention mechanism of the graph attention network implemented by the edge fusion graph attention module is also fused by edge features before being multiplied by attention scores weighted by a cosine cutoff as shown in Eq. 12,

$${α}_{ij}^{l} = σ ( ( {W}_{Q}^{l} {h}_{i}^{l} ) {( {W}_{K}^{l} {h}_{j}^{l} ⊙ {Dense}_{K}^{l} ( {f}_{ij}^{l} ) )}^{T} ) \qquad ( 11 )$$

$${m}_{ij}^{l} = {α}_{ij}^{l} \cdot ϕ ( ‖ {\vec{r}}_{ij} ‖ ) \cdot ( {W}_{V}^{l} {h}_{j}^{l} ⊙ {Dense}_{V}^{l} ( {f}_{ij}^{l} ) ) \qquad ( 12 )$$

where l∈{0, 1, 2, . . . , L} is the index of block, σ denotes the activation function (e.g., SiLU), W is the learnable weight matrix, ⊙ represents the Hadamard product, ϕ(·) denotes the cosine cutoff and Dense(·) refers to one learnable weight matrix with activation function. For brevity, the learnable bias is omitted for linear transformation on scalar embedding in equations, and there is no bias for vector embedding to ensure universal equivariance. Equations 11 and 12 are shown in graphical form in FIG. 9B, with the output of the Equation 12 being passed to the downstream dense layer.

Returning to FIG. 9A, then, the computed

$${m}_{ij}^{l}$$

is used to produce the geometric messages

$${\vec{m}}_{ij}^{l}$$

for vectors:

$${\vec{m}}_{ij}^{l} = ( {Dense}_{u}^{l} ( {m}_{ij}^{l} ) ⊙ {\vec{u}}_{ij} ) + ( {Dense}_{v}^{l} ( {m}_{ij}^{l} ) ⊙ {\vec{v}}_{j}^{l} ) \qquad ( 13 )$$

And the vector embedding \vec{v}^{l} is updated by:

$${m}_{ij}^{l} = {\sum }_{j ∈ N ( i )} {m}_{ij}^{l} , \qquad ( 14 )$$

$${\vec{m}}_{ij}^{l} = {\sum }_{j ∈ N ( i )} {\vec{m}}_{ij}^{l}$$

$$Δ {\vec{v}}_{i}^{l + 1} = {\vec{m}}_{i}^{l} + {W}_{vm}^{l} {m}_{i}^{l} ⊙ {W}_{v}^{l} {\vec{v}}_{i}^{l} \qquad ( 15 )$$

Continuing with FIG. 9A, in the Vec2Scalar module, the node embedding

$${h}_{i}^{l}$$

and edge embedding

$${f}_{ij}^{l}$$

are updated by the geometric information extracted by the RGC strategy, i.e., angles (Eq. 7) and dihedrals (Eq. 8), respectively. The residual node embedding

$$Δ {h}_{i}^{l + 1}$$

is calculated by a Hadamard product between the runtime angle information and the aggregated scalar messages with a gated residual connection:

$$Δ {h}_{i}^{l + 1} = 〈 {W}_{t}^{l} {\vec{v}}_{i}^{l} , {W}_{s}^{l} {\vec{v}}_{i}^{l} 〉 ⊙ {W}_{Angle}^{l} {m}_{i}^{l} + {W}_{res}^{l} {m}_{i}^{l} \qquad ( 16 )$$

To compute the residual edge embedding

$$Δ {f}_{ij}^{l + 1} ,$$

the Hadamard product of the runtime dihedral information with the transformed edge embedding is performed:

$$Δ {f}_{ij}^{l + 1} = 〈 Re {j}_{{\vec{r}}_{ij}} ( {W}_{Rt}^{l} {\vec{v}}_{i}^{l} ) , Re {j}_{{\vec{r}}_{ji}} ( {W}_{Rs}^{l} {\vec{v}}_{j}^{l} ) 〉 ⊙ {Dense}_{Dihedral}^{l} ( {f}_{ij}^{l} ) \qquad ( 17 )$$

After the residual hidden representations are calculated, the residual hidden representations are added to the original input of block/and feed them to the next block.

In the output block, the scalar embedding and vector embedding of nodes are updated with multiple gated equivariant blocks:

$${t}_{i}^{l} = {Dense}_{{O}_{2}}^{l} ( [ ‖ {W}_{{O}_{1}}^{l} {\vec{v}}_{i}^{l} ‖ , {h}_{l}^{i} ] ) \qquad ( 18 )$$

$${h}_{i}^{l + 1} = {W}_{{O}_{3}}^{l} {t}_{i}^{l} \qquad ( 19 )$$

$${\vec{v}}_{i}^{l + 1} = {W}_{{O}_{4}}^{l} {\vec{v}}_{i}^{l} ⊙ {W}_{{O}_{5}}^{l} {t}_{i}^{l} \qquad ( 20 )$$

where [·, ·] is the tensor concatenation operation. The final scalar embedding

$${h}_{i}^{L}$$

∈ [symbol] and vector embedding

$${\vec{v}}_{i}^{L}$$

∈ [symbol] are used to predict various molecular properties.

On QM9, the molecular dipole is calculated as follows:

$$μ = ‖ {\sum }_{i = 1}^{N} {\vec{v}}_{i}^{L} + {h}_{i}^{L} ( {\vec{r}}_{i} - {\vec{r}}_{c} ) ‖ \qquad ( 21 )$$

where \vec{r}_{c} denotes the center of mass. Similarly, for the prediction of electronic spatial extent [symbol] R² [symbol] , the following equation is used:

$$〈 {R}^{2} 〉 = {\sum }_{i = 1}^{N} {h}_{i}^{L} {‖ {\vec{r}}_{i} - {\vec{r}}_{c} ‖}^{2} \qquad ( 22 )$$

For the remaining 10 properties y, the final scalar embedding of nodes is aggregated as follows:

$$y = {\sum }_{i = 1}^{N} {h}_{i}^{L} \qquad ( 23 )$$

For models trained on the molecular dynamics datasets including MD17, revised MD17, and Chignolin, the total potential energy is obtained as the sum of the final scalar embedding of the nodes. As an energy-conserving potential, the forces are then calculated using the negative gradients of the predicted total potential energy with respect to the atomic coordinates:

$$E = {\sum }_{i = 1}^{N} {h}_{i}^{L} \qquad ( 24 )$$

$${\vec{F}}_{i} = - {∇}_{i} E \qquad ( 25 )$$

The design of Chignolin dataset will now be described. The initial structure for Replica Exchange Molecular Dynamics (REMD) simulations is derived from a protein data bank (PDB ID: 5AWL). Water molecules in the crystal structure are removed. Then, FF19SB force field is applied to describe the atomic interactions for Chignolin in a generalized Born implicit solvent model. A second modification of the Bondi Van der Waals radii set is used in the solvent model. The program CHIR_RST in Amber 20 is applied to create chiral restraint file during REMD simulation to maintain the chiral property at a high temperature. The system at the beginning encountered a minimization process of 500 steepest descent and 500 conjugate gradient cycles. After energy minimization, 200 ps of equilibration runs at 300 K, 400 K, 500 K, 600 K, 700 K, 800 K, 900 K, 1000 K were applied to the system with random initial velocities. The final structure of equilibration was used for REMD simulations at the corresponding temperatures. Each single replica in the production ran for 2 ps and then was exchanged to the neighboring temperature. The exchange happened 5,000 times in each production run, and 8 replica temperatures are obtained, which led to a total simulation time of 80 ns. The sampling interval of each simulation trajectory is 0.4 ps so the trajectory had 200,000 points. 10,000 points are evenly picked from the REMD trajectory to generate the input file for Gaussian 16. The potential energy and the atomic forces for each conformation were calculated with M06-2X functional and 6-31G* basis. The integration grid was set to superfine precision.

Finally, 9,543 SCF converged conformations with the total potential energy and atomic forces were recruited from the Chignolin dataset. The distribution of the total energy ranged from-2,831,076.155 kcal/mol to-2,830,477.983 kcal/-mol, and some representative conformations are shown in Supplementary FIG. 2. Note that the total energy does not show a normal distribution, but has two peaks corresponding to the folded and unfolded states of Chignolin, which increased the difficulty for model training on the dataset.

Regarding data splitting schemes, for the QM9 dataset, the dataset was randomly split into 110,000 samples as the train set, 10,000 samples as the validation set, and the rest as the test set by following the previous studies. To evaluate the effectiveness of ViSNet to simulation data, ViSNet was trained on MD17 and rMD17 with a limited data setting, which consists of only 950 uniformly sampled conformations for model training and 50 conformations for validation for each molecule.

Furthermore, the whole Chignolin dataset was randomly split into 80%, 10%, and 10% as the training, validation, and test datasets. Six representative conformations are picked from the test set for illustration.

Regarding experimental settings, for the QM9 dataset a batch size of 32 and a learning rate of 1e-4 for all the properties was adopted. The mean squared error (MSE) loss was used for model training. For the molecular dynamic dataset including MD17, rMD17, and Chignolin, a combined MSE loss for energy and force prediction was leveraged. The weight of energy loss is set to 0.05 for MD17 and rMD17, 0.2 for Chignolin. The weight of forces loss was set to 0.95 for MD17 and rMD17, 0.8 for Chignolin. The batch size was set to 4 and the learning rate is chosen from 2e-4, 3e-4, 4e-4 for different molecules. The cutoff was set to 5 for small molecules in QM9, MD17, and rMD17 and changed to 4 for Chignolin in order to reduce the number of edges in the molecular graphs. The learning rate decay was used if the validation loss stopped decreasing. The patience was set to 15 epochs for QM9, and 30 epochs for MD17, rMD17, and Chignolin. The learning rate decay factor was set to 0.8 for these models. An early stopping strategy was adopted to prevent over-fitting. The ViSNet model trained on the molecular dynamic datasets had 9 hidden layers and the embedding dimension was set to 256. A larger model was used for the QM9 dataset, i.e., the embedding dimension changed to 512. Experiments were conducted on NVIDIA® 32G-V100 GPUS.

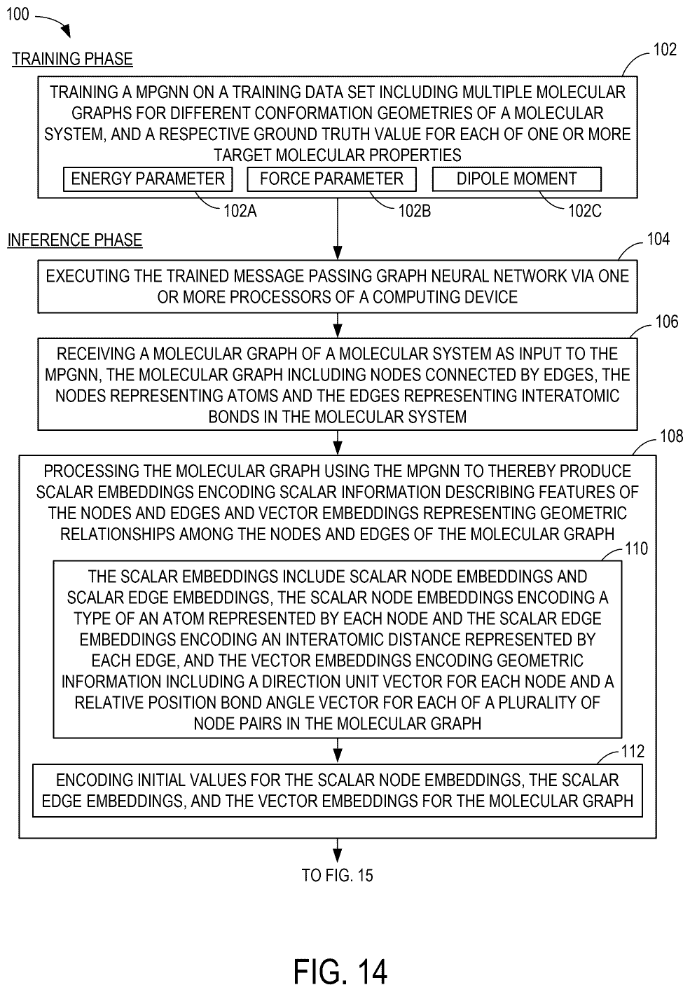

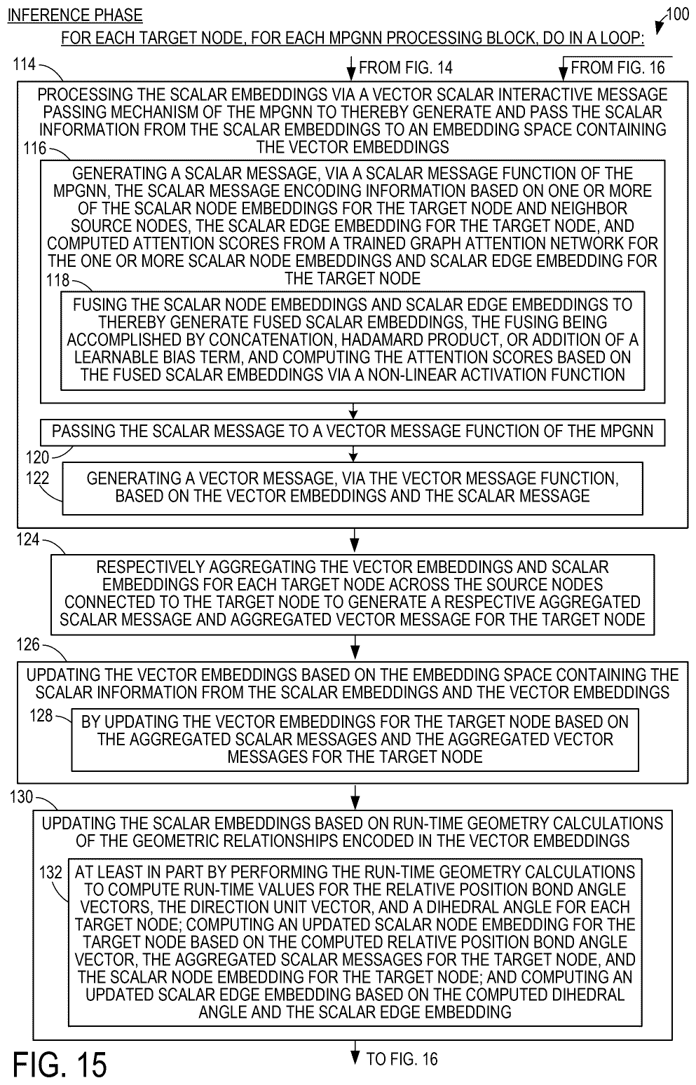

FIGS. 14-16 show a flowchart of a computerized method 100 according to one example implementation of the present disclosure. 100 may be implemented by the hardware and software of computing system 10 described above, or by other suitable hardware and software.

At 102, method 100 includes during a training phase prior to an inference phase, training a message passing graph neural network (MPGNN) on a training data set including multiple molecular graphs for different conformation geometries of a molecular system, and a respective ground truth value for a target molecular property for each molecular graph. As shown, the target molecular property may be an energy parameter 102A, a force parameter 102B, or a dipole moment 102C. Other parameters are also contemplated as discussed above.

At 104, method 100 includes executing a message passing graph neural network (MPGNN) via one or more processors of a computing device. At 106, the method 100 further includes receiving a molecular graph of a molecular system as input to the MPGNN. The molecular graph typically includes nodes connected by edges, the nodes representing atoms and the edges representing interatomic bonds in the molecular system.

At 108, the method 100 includes processing the molecular graph using the MPGNN to thereby produce scalar embeddings encoding scalar information describing features of the nodes and edges and vector embeddings representing geometric relationships among the nodes and edges of the molecular graph. As shown at 110, the scalar embeddings can include scalar node embeddings and scalar edge embeddings. The scalar node embeddings can encode a type of an atom represented by each node and the scalar edge embeddings can encode an interatomic distance represented by each edge. The vector embeddings can encode geometric information including a direction unit vector for each node and a relative position bond angle vector for each of a plurality of node pairs in the molecular graph. At 112, processing the molecular graph at 108 is shown to include encoding initial values for the scalar node embeddings, the scalar edge embeddings, and the vector embeddings for the molecular graph.

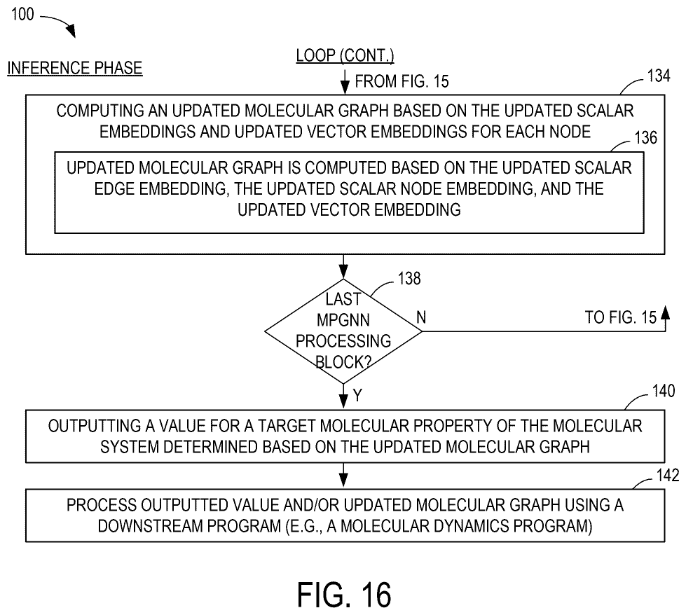

Continuing from step 112 in FIG. 14 to step 114 in FIG. 15, a MPGNN processing block loop is illustrated that begins at step 114 and continues until step 138 in FIG. 16, the method 100 loops back until the last MPGNN processing block is reached. Thus, the method 100 may further for each MPGNN processing block, for each of a plurality of target nodes in the molecular graph, in the MPGNN processing block loop, performing steps 114-138.

At 114, method 100 includes processing the scalar embeddings via a vector scalar interactive message passing mechanism of the MPGNN to thereby generate and pass the scalar information from the scalar embeddings to an embedding space containing the vector embeddings. Substeps of processing at 114 are illustrated at 116-122. At 116, processing the scalar embeddings via the vector scalar interactive message passing mechanism at 114 is shown to be accomplished at least in part by generating a scalar message, via a scalar message function of the MPGNN, the scalar message encoding information based on one or more of the scalar node embeddings for the target node and neighbor source nodes, the scalar edge embedding for the target node, and computed attention scores from a trained graph attention network for the one or more scalar node embeddings and scalar edge embedding for the target node. At 118, it is shown that generating the scalar message can be accomplished at least in part by fusing the scalar node embeddings and scalar edge embeddings to thereby generate fused scalar embeddings, the fusing being accomplished by concatenation, Hadamard product, or addition of a learnable bias term, and computing the attention scores based on the fused scalar embeddings via a non-linear activation function. Other techniques for fusing the embeddings may also be applied, as well as other activations functions that preserve equivariance.

Processing the scalar embeddings at 114 can include, at 120 passing the scalar message to a vector message function of the MPGNN, and, at 122, generating a vector message, via the vector message function, based on the vector embeddings and the scalar message.

At 124, in the MPGNN processing block loop, the method 100 further includes, prior to updating the vector embeddings and scalar embeddings, respectively aggregating the vector embeddings and scalar embeddings for each target node across the source nodes connected to the target node to generate a respective aggregated scalar message and aggregated vector message for the target node.

At 126, method 100 includes updating the vector embeddings based on the embedding space containing the scalar information from the scalar embeddings and the vector embeddings. At 128, updating the vector embeddings is accomplished at least in part by updating the vector embeddings for the target node based on the aggregated scalar messages and the aggregated vector messages for the target node.

At 134, method 100 includes updating the scalar embeddings based on run-time geometry calculations of the geometric relationships encoded in the vector embeddings. At 132, updating the scalar embeddings is accomplished at least in part by performing the run-time geometry calculations to compute run-time values for the relative position bond angle vectors, the direction unit vector, and a dihedral angle for the target node; computing an updated scalar node embedding for the target node based on the computed relative position bond angle vector, the aggregated scalar messages for the target node, and the scalar node embedding for the target node; and computing an updated scalar edge embedding based on the computed dihedral angle and the scalar edge embedding.

At 134, method 100 includes computing an updated molecular graph based on the updated scalar embeddings and updated vector embeddings for each node. At 136, the updated molecular graph is computed based on the updated scalar edge embedding, the updated scalar node embedding, and the updated vector embedding.

At 138, the method 100 includes determining if the last MPGNN processing block has been completed, and if so, the method proceeds to step 140. Otherwise, if not, the method 100 loops back to step 114 in FIG. 15. At 140, method 100 includes outputting a value for a target molecular property of the molecular system determined based on the updated molecular graph. At 142, the method can include processing the outputted value for the target molecular property and/or outputted updated molecular graph using a downstream program, such as a molecular dynamics program, as discussed above.

The systems and methods described herein have the demonstrated technical benefits of increased accuracy with decreased computational costs over state of the art models, as discussed above.

In some embodiments, the methods and processes described herein may be tied to a computing system of one or more computing devices. In particular, such methods and processes may be implemented as a computer-application program or service, an application-programming interface (API), a library, and/or other computer-program product.

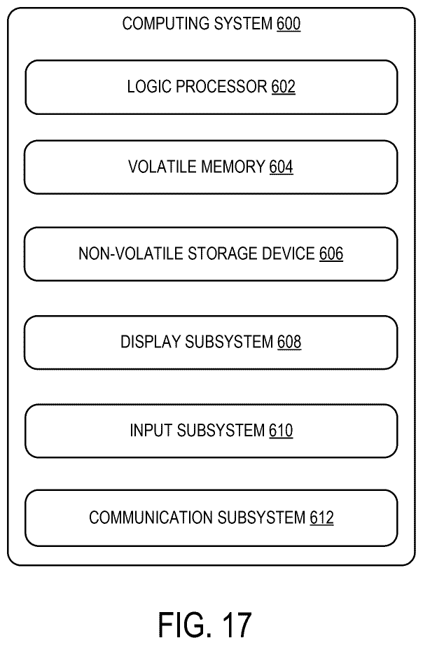

FIG. 17 schematically shows a non-limiting embodiment of a computing system 600 that can enact one or more of the methods and processes described above. Computing system 600 is shown in simplified form. Computing system 600 may embody the computer system 10 described above and illustrated in FIG. 1. Computing system 600 may take the form of one or more personal computers, server computers, tablet computers, home-entertainment computers, network computing devices, gaming devices, mobile computing devices, mobile communication devices (e.g., smart phone), and/or other computing devices, and wearable computing devices such as smart wristwatches and head mounted augmented reality devices.

Computing system 600 includes a logic processor 602 volatile memory 604, and a non-volatile storage device 606. Computing system 600 may optionally include a display subsystem 608, input subsystem 610, communication subsystem 612, and/or other components not shown in FIG. 17.

Logic processor 602 includes one or more physical devices configured to execute instructions. For example, the logic processor may be configured to execute instructions that are part of one or more applications, programs, routines, libraries, objects, components, data structures, or other logical constructs. Such instructions may be implemented to perform a task, implement a data type, transform the state of one or more components, achieve a technical effect, or otherwise arrive at a desired result.

The logic processor may include one or more physical processors (hardware) configured to execute software instructions. Additionally or alternatively, the logic processor may include one or more hardware logic circuits or firmware devices configured to execute hardware-implemented logic or firmware instructions. Processors of the logic processor 602 may be single-core or multi-core, and the instructions executed thereon may be configured for sequential, parallel, and/or distributed processing. Individual components of the logic processor optionally may be distributed among two or more separate devices, which may be remotely located and/or configured for coordinated processing. Aspects of the logic processor may be virtualized and executed by remotely accessible, networked computing devices configured in a cloud-computing configuration. In such a case, these virtualized aspects are run on different physical logic processors of various different machines, it will be understood.

Non-volatile storage device 606 includes one or more physical devices configured to hold instructions executable by the logic processors to implement the methods and processes described herein. When such methods and processes are implemented, the state of non-volatile storage device 606 may be transformed—e.g., to hold different data.

Non-volatile storage device 606 may include physical devices that are removable and/or built in. Non-volatile storage device 606 may include optical memory (e.g., CD, DVD, HD-DVD, Blu-Ray Disc, etc.), semiconductor memory (e.g., ROM, EPROM, EEPROM, FLASH memory, etc.), and/or magnetic memory (e.g., hard-disk drive, floppy-disk drive, tape drive, MRAM, etc.), or other mass storage device technology. Non-volatile storage device 606 may include nonvolatile, dynamic, static, read/write, read-only, sequential-access, location-addressable, file-addressable, and/or content-addressable devices. It will be appreciated that non-volatile storage device 606 is configured to hold instructions even when power is cut to the non-volatile storage device 606.

Volatile memory 604 may include physical devices that include random access memory. Volatile memory 604 is typically utilized by logic processor 602 to temporarily store information during processing of software instructions. It will be appreciated that volatile memory 604 typically does not continue to store instructions when power is cut to the volatile memory 604.

Aspects of logic processor 602, volatile memory 604, and non-volatile storage device 606 may be integrated together into one or more hardware-logic components. Such hardware-logic components may include field-programmable gate arrays (FPGAs), program-and application-specific integrated circuits (PASIC/ASICs), program-and application-specific standard products (PSSP/ASSPs), system-on-a-chip (SOC), and complex programmable logic devices (CPLDs), for example.

The terms “module,” “program,” and “engine” may be used to describe an aspect of computing system 600 typically implemented in software by a processor to perform a particular function using portions of volatile memory, which function involves transformative processing that specially configures the processor to perform the function. Thus, a module, program, or engine may be instantiated via logic processor 602 executing instructions held by non-volatile storage device 606, using portions of volatile memory 604. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

When included, display subsystem 608 may be used to present a visual representation of data held by non-volatile storage device 606. The visual representation may take the form of a graphical user interface (GUI). As the herein described methods and processes change the data held by the non-volatile storage device, and thus transform the state of the non-volatile storage device, the state of display subsystem 608 may likewise be transformed to visually represent changes in the underlying data. Display subsystem 608 may include one or more display devices utilizing virtually any type of technology. Such display devices may be combined with logic processor 602, volatile memory 604, and/or non-volatile storage device 606 in a shared enclosure, or such display devices may be peripheral display devices.

When included, input subsystem 610 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on-or off-board. Example NUI componentry may include a microphone for speech and/or voice recognition; an infrared, color, stereoscopic, and/or depth camera for machine vision and/or gesture recognition; a head tracker, eye tracker, accelerometer, and/or gyroscope for motion detection and/or intent recognition; as well as electric-field sensing componentry for assessing brain activity; and/or any other suitable sensor. When included, communication subsystem 612 may be configured to communicatively couple various computing devices described herein with each other, and with other devices. Communication subsystem 612 may include wired and/or wireless communication devices compatible with one or more different communication protocols. As non-limiting examples, the communication subsystem may be configured for communication via a wireless telephone network, or a wired or wireless local- or wide-area network, such as a HDMI over Wi-Fi connection. In some embodiments, the communication subsystem may allow computing system 600 to send and/or receive messages to and/or from other devices via a network such as the Internet.

The following paragraphs discuss several aspects of the present disclosure. According to one aspect of the present disclosure, a computing system is provided. The system may include one or more processors configured to, during an inference phase, execute a message passing graph neural network (MPGNN) including an embedding block, one or more MPGNN processing blocks each including a respective message passing sub-block and a respective update sub-block, and an output block, wherein the message passing sub-block is configured with a vector scalar interactive message passing mechanism. The processor may be further configured to receive a molecular graph of a molecular system as input to the MPGNN, the molecular graph including nodes connected by edges, the nodes representing atoms and the edges representing interatomic bonds in the molecular system. The processor may be further configured to process the molecular graph using the embedding block to thereby produce scalar embeddings encoding scalar information describing features of the nodes and edges and vector embeddings representing geometric relationships among the nodes and edges of the molecular graph. The processor may be further configured to process the scalar embeddings via the vector scalar interactive message passing mechanism of the message passing sub-block of the MPGNN to thereby generate and pass the scalar information from the scalar embeddings to an embedding space containing the vector embeddings. The processor may be further configured to update, via the update sub-block, the vector embeddings based on the embedding space containing the scalar information from the scalar embeddings and the vector embeddings. The processor may be further configured to update, via the update sub-block, the scalar embeddings based on run-time geometry calculations of the geometric relationships encoded in the vector embeddings. The processor may be further configured to compute, via the update sub-block, an updated molecular graph based on the updated scalar embeddings and updated vector embeddings for each node. The processor may be further configured to output, via the output block, a value for a target molecular property of the molecular system determined based on the updated molecular graph.

According to this aspect, the scalar embeddings may include scalar node embeddings and scalar edge embeddings, the scalar node embeddings encoding a type of an atom represented by each node and the scalar edge embeddings encoding an interatomic distance represented by each edge, and the vector embeddings encoding geometric information including a direction unit vector for each node and a relative position bond angle vector for each of a plurality of node pairs in the molecular graph, and processing the molecular graph using the embedding block may include encoding, via the embedding block, initial values for the scalar node embeddings, the scalar edge embeddings, and the vector embeddings for the molecular graph.

According to this aspect, the processor may be configured to process the scalar embeddings via the vector scalar interactive message passing mechanism of the message passing sub-block at least in part by, for each MPGNN processing block, for each of a plurality of target nodes in the molecular graph, in a MPGNN processing block loop, generating a scalar message, via a scalar message function of the message passing sub-block, the scalar message encoding information based on one or more of the scalar node embeddings for the target node and neighbor source nodes, the scalar edge embedding for the target node, and computed attention scores from a trained graph attention network for the one or more scalar node embeddings and scalar edge embedding for the target node, passing the scalar message to a vector message function of the message passing sub-block, and generating a vector message, via the vector message function, based on the vector embeddings and the scalar message.

According to this aspect, the processor may be configured to generate the scalar message, at least in part by fusing the scalar node embeddings and scalar edge embeddings to thereby generate fused scalar embeddings, in which the fusing may be accomplished by concatenation, Hadamard product, or addition of a learnable bias term, and computing the attention scores based on the fused scalar embeddings via a non-linear activation function.

According to this aspect, in the MPGNN processing block loop, the processor may be configured to, prior to updating the vector embeddings and scalar embeddings, respectively aggregate the vector embeddings and scalar embeddings for each target node across the source nodes connected to the target node to generate a respective aggregated scalar message and aggregated vector message for the target node.

According to this aspect, in the MPGNN processing block loop, the processor may be configured to update, via the update sub-block, the vector embeddings at least in part by updating, via the update sub-block, the vector embeddings for the target node based on the aggregated scalar messages and the aggregated vector messages for the target node.

According to this aspect, in the MPGNN processing block loop, the processor may be configured to update, via the update sub-block, the scalar embeddings at least in part by performing, via the update sub-block, the run-time geometry calculations to compute run-time values for the relative position bond angle vectors, the direction unit vector, and a dihedral angle for each target node, computing, via the update sub-block, an updated scalar node embedding for the target node based on the computed relative position bond angle vector, the aggregated scalar messages for the target node, and the scalar node embedding for the target node, and computing, via the update sub-block, an updated scalar edge embedding based on the computed dihedral angle and the scalar edge embedding.

According to this aspect, the processor may be configured to compute the updated molecular graph based on the updated scalar edge embedding, the updated scalar node embedding, and the updated vector embedding.

According to this aspect, the processor may be further configured to, during a training phase prior to the inference phase, train the MPGNN on a training data set including multiple molecular graphs for different conformation geometries of the molecular system, and a respective ground truth value for the target molecular property for each molecular graph.

According to this aspect, the ground truth value may be computed via density functional theory.

According to this aspect, the target molecular property may be an energy parameter, a force parameter, or a dipole moment.

According to this aspect, the value for the target molecular property may be output to a molecular dynamics simulation program for use in a molecular dynamics simulation.

According to another aspect of the present disclosure, a computerized method is provided. The computerized method may include, executing a message passing graph neural network (MPGNN) via one or more processors of a computing device. The computerized method may further include receiving a molecular graph of a molecular system as input to the MPGNN, the molecular graph including nodes connected by edges, the nodes representing atoms and the edges representing interatomic bonds in the molecular system. The computerized method may further include processing the molecular graph using the MPGNN to thereby produce scalar embeddings encoding scalar information describing features of the nodes and edges and vector embeddings representing geometric relationships among the nodes and edges of the molecular graph. The computerized method may further include processing the scalar embeddings via a vector scalar interactive message passing mechanism of the MPGNN to thereby generate and pass the scalar information from the scalar embeddings to an embedding space containing the vector embeddings. The computerized method may further include updating the vector embeddings based on the embedding space containing the scalar information from the scalar embeddings and the vector embeddings. The computerized method may further include updating the scalar embeddings based on run-time geometry calculations of the geometric relationships encoded in the vector embeddings. The computerized method may further include computing an updated molecular graph based on the updated scalar embeddings and updated vector embeddings for each node. The computerized method may further include outputting a value for a target molecular property of the molecular system determined based on the updated molecular graph.

According to this aspect, the scalar embeddings may include scalar node embeddings and scalar edge embeddings, the scalar node embeddings encoding a type of an atom represented by each node and the scalar edge embeddings encoding an interatomic distance represented by each edge, and the vector embeddings encoding geometric information including a direction unit vector for each node and a relative position bond angle vector for each of a plurality of node pairs in the molecular graph, and processing the molecular graph may include encoding initial values for the scalar node embeddings, the scalar edge embeddings, and the vector embeddings for the molecular graph.

According to this aspect, processing the scalar embeddings via the vector scalar interactive message passing mechanism may be accomplished at least in part by, for each MPGNN processing block, for each of a plurality of target nodes in the molecular graph, in a MPGNN processing block loop, generating a scalar message, via a scalar message function of the MPGNN, the scalar message encoding information based on one or more of the scalar node embeddings for the target node and neighbor source nodes, the scalar edge embedding for the target node, and computed attention scores from a trained graph attention network for the one or more scalar node embeddings and scalar edge embedding for the target node, passing the scalar message to a vector message function of the MPGNN, and generating a vector message, via the vector message function, based on the vector embeddings and the scalar message.

According to this aspect, generating the scalar message may be accomplished at least in part by fusing the scalar node embeddings and scalar edge embeddings to thereby generate fused scalar embeddings, in which the fusing may be accomplished by concatenation, Hadamard product, or addition of a learnable bias term, and computing the attention scores based on the fused scalar embeddings via a non-linear activation function, and in the MPGNN processing block loop, the computerized method may further include, prior to updating the vector embeddings and scalar embeddings, respectively aggregating the vector embeddings and scalar embeddings for each target node across the source nodes connected to the target node to generate a respective aggregated scalar message and aggregated vector message for the target node.

According to this aspect, in the MPGNN processing block loop, updating the vector embeddings may be accomplished at least in part by updating the vector embeddings for the target node based on the aggregated scalar messages and the aggregated vector messages for the target node, and updating the scalar embeddings may be accomplished at least in part by performing the run-time geometry calculations to compute run-time values for the relative position bond angle vectors, the direction unit vector, and a dihedral angle for each target node; computing an updated scalar node embedding for the target node based on the computed relative position bond angle vector, the aggregated scalar messages for the target node, and the scalar node embedding for the target node; and computing an updated scalar edge embedding based on the computed dihedral angle and the scalar edge embedding, in which the updated molecular graph may be computed based on the updated scalar edge embedding, the updated scalar node embedding, and the updated vector embedding.

According to this aspect, the computerized method may further include, during a training phase prior to the inference phase, training the MPGNN on a training data set including multiple molecular graphs for different conformation geometries of the molecular system, and a respective ground truth value for the target molecular property for each molecular graph, in which the target molecular property may be an energy parameter, a force parameter, or a dipole moment.

According to another aspect of the present disclosure, a computing system is provided. The system may include one or more processors configured to, during an inference phase, execute a message passing graph neural network (MPGNN) including an embedding block, one or more MPGNN processing blocks each including a respective message passing sub-block and a respective update sub-block, and an output block, wherein the message passing sub-block is configured with a vector scalar interactive message passing mechanism. The processor may be further configured to The processor may be further configured to receive a molecular graph of a molecular system as input to the MPGNN, the molecular graph including nodes connected by edges, the nodes representing atoms and the edges representing interatomic bonds in the molecular system. The processor may be further configured to process the molecular graph using the embedding block to thereby produce scalar embeddings encoding scalar information describing features of the nodes and edges and vector embeddings representing geometric relationships among the nodes and edges of the molecular graph. The processor may be further configured to update, via the update sub-block, the vector embeddings based on the scalar information from the scalar embeddings and the vector embeddings. The processor may be further configured to update, via the update sub-block, the scalar embeddings based on run-time geometry calculations of the geometric relationships encoded in the vector embeddings. The processor may be further configured to compute, via the update sub-block, an updated molecular graph based on the updated scalar embeddings and updated vector embeddings for each node. The processor may be further configured to output, via the output block, a value for a target molecular property of the molecular system determined based on the updated molecular graph.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A computing system, comprising:
one or more processors configured to:
during an inference phase,
execute a message passing graph neural network (MPGNN) including an embedding block, one or more MPGNN processing blocks each including a respective message passing sub-block and a respective update sub-block, and an output block, wherein the message passing sub-block is configured with a vector scalar interactive message passing mechanism;
receive a molecular graph of a molecular system as input to the MPGNN, the molecular graph including nodes connected by edges, the nodes representing atoms and the edges representing interatomic bonds in the molecular system;
process the molecular graph using the embedding block to thereby produce scalar embeddings encoding scalar information describing features of the nodes and edges and vector embeddings representing geometric relationships among the nodes and edges of the molecular graph;
process the scalar embeddings via the vector scalar interactive message passing mechanism of the message passing sub-block of the MPGNN to thereby generate and pass the scalar information from the scalar embeddings to an embedding space containing the vector embeddings;
update, via the update sub-block, the vector embeddings based on the embedding space containing the scalar information from the scalar embeddings and the vector embeddings;
update, via the update sub-block, the scalar embeddings based on run-time geometry calculations of the geometric relationships encoded in the vector embeddings;
compute, via the update sub-block, an updated molecular graph based on the updated scalar embeddings and updated vector embeddings for each node; and
output, via the output block, a value for a target molecular property of the molecular system determined based on the updated molecular graph.

2. The computing system of claim 1, wherein
the scalar embeddings include scalar node embeddings and scalar edge embeddings, the scalar node embeddings encoding a type of an atom represented by each node and the scalar edge embeddings encoding an interatomic distance represented by each edge, and the vector embeddings encoding geometric information including a direction unit vector for each node and a relative position bond angle vector for each of a plurality of node pairs in the molecular graph; and
processing the molecular graph using the embedding block includes encoding, via the embedding block, initial values for the scalar node embeddings, the scalar edge embeddings, and the vector embeddings for the molecular graph.

3. The computing system of claim 2, wherein
the processor is configured to process the scalar embeddings via the vector scalar interactive message passing mechanism of the message passing sub-block at least in part by:
for each MPGNN processing block, for each of a plurality of target nodes in the molecular graph, in a MPGNN processing block loop:
generating a scalar message, via a scalar message function of the message passing sub-block, the scalar message encoding information based on one or more of the scalar node embeddings for the target node and neighbor source nodes, the scalar edge embedding for the target node, and computed attention scores from a trained graph attention network for the one or more scalar node embeddings and scalar edge embedding for the target node;
passing the scalar message to a vector message function of the message passing sub-block; and
generating a vector message, via the vector message function, based on the vector embeddings and the scalar message.

4. The computing system of claim 3, wherein the processor is configured to generate the scalar message, at least in part by:
fusing the scalar node embeddings and scalar edge embeddings to thereby generate fused scalar embeddings, the fusing being accomplished by concatenation, Hadamard product, or addition of a learnable bias term, and computing the attention scores based on the fused scalar embeddings via a non-linear activation function.

5. The computing system of claim 3, wherein, in the MPGNN processing block loop, the processor is configured to:
prior to updating the vector embeddings and scalar embeddings, respectively aggregate the vector embeddings and scalar embeddings for each target node across the source nodes connected to the target node to generate a respective aggregated scalar message and aggregated vector message for the target node.

6. The computing system of claim 5, wherein, in the MPGNN processing block loop, the processor is configured to update, via the update sub-block, the vector embeddings at least in part by:
updating, via the update sub-block, the vector embeddings for the target node based on the aggregated scalar messages and the aggregated vector messages for the target node.

7. The computing system of claim 6, wherein, in the MPGNN processing block loop, the processor is configured to update, via the update sub-block, the scalar embeddings at least in part by:
performing, via the update sub-block, the run-time geometry calculations to compute run-time values for the relative position bond angle vectors, the direction unit vector, and a dihedral angle for each target node;
computing, via the update sub-block, an updated scalar node embedding for the target node based on the computed relative position bond angle vector, the aggregated scalar messages for the target node, and the scalar node embedding for the target node; and
computing, via the update sub-block, an updated scalar edge embedding based on the computed dihedral angle and the scalar edge embedding.

8. The computing system of claim 7, wherein the processor is configured to compute the updated molecular graph based on the updated scalar edge embedding, the updated scalar node embedding, and the updated vector embedding.

9. The computing system of claim 1, wherein the processor is further configured to:
during a training phase prior to the inference phase,
train the MPGNN on a training data set including multiple molecular graphs for different conformation geometries of the molecular system, and a respective ground truth value for the target molecular property for each molecular graph.

10. The computing system of claim 9, wherein the ground truth value is computed via density functional theory.

11. The computing system of claim 1, wherein the target molecular property is an energy parameter, a force parameter, or a dipole moment.

12. The computing system of claim 1, wherein the value for the target molecular property is output to a molecular dynamics simulation program for use in a molecular dynamics simulation.

13. A computerized method, comprising:
executing a message passing graph neural network (MPGNN) via one or more processors of a computing device:
receiving a molecular graph of a molecular system as input to the MPGNN, the molecular graph including nodes connected by edges, the nodes representing atoms and the edges representing interatomic bonds in the molecular system;
processing the molecular graph using the MPGNN to thereby produce scalar embeddings encoding scalar information describing features of the nodes and edges and vector embeddings representing geometric relationships among the nodes and edges of the molecular graph;
processing the scalar embeddings via a vector scalar interactive message passing mechanism of the MPGNN to thereby generate and pass the scalar information from the scalar embeddings to an embedding space containing the vector embeddings;
updating the vector embeddings based on the embedding space containing the scalar information from the scalar embeddings and the vector embeddings;
updating the scalar embeddings based on run-time geometry calculations of the geometric relationships encoded in the vector embeddings;
computing an updated molecular graph based on the updated scalar embeddings and updated vector embeddings for each node; and
outputting a value for a target molecular property of the molecular system determined based on the updated molecular graph.

14. The computerized method of claim 13, wherein
the scalar embeddings include scalar node embeddings and scalar edge embeddings, the scalar node embeddings encoding a type of an atom represented by each node and the scalar edge embeddings encoding an interatomic distance represented by each edge, and the vector embeddings encoding geometric information including a direction unit vector for each node and a relative position bond angle vector for each of a plurality of node pairs in the molecular graph; and
processing the molecular graph includes encoding initial values for the scalar node embeddings, the scalar edge embeddings, and the vector embeddings for the molecular graph.

15. The computerized method of claim 14, wherein
processing the scalar embeddings via the vector scalar interactive message passing mechanism is accomplished at least in part by:
for each MPGNN processing block, for each of a plurality of target nodes in the molecular graph, in a MPGNN processing block loop:
generating a scalar message, via a scalar message function of the MPGNN, the scalar message encoding information based on one or more of the scalar node embeddings for the target node and neighbor source nodes, the scalar edge embedding for the target node, and computed attention scores from a trained graph attention network for the one or more scalar node embeddings and scalar edge embedding for the target node;
passing the scalar message to a vector message function of the MPGNN; and
generating a vector message, via the vector message function, based on the vector embeddings and the scalar message.

16. The computerized method of claim 15, wherein generating the scalar message is accomplished at least in part by:
fusing the scalar node embeddings and scalar edge embeddings to thereby generate fused scalar embeddings, the fusing being accomplished by concatenation, Hadamard product, or addition of a learnable bias term, and computing the attention scores based on the fused scalar embeddings via a non-linear activation function; and
wherein, in the MPGNN processing block loop, the method further includes, prior to updating the vector embeddings and scalar embeddings, respectively aggregating the vector embeddings and scalar embeddings for each target node across the source nodes connected to the target node to generate a respective aggregated scalar message and aggregated vector message for the target node.

17. The computerized method of claim 16, wherein, in the MPGNN processing block loop,
updating the vector embeddings is accomplished at least in part by updating the vector embeddings for the target node based on the aggregated scalar messages and the aggregated vector messages for the target node; and
updating the scalar embeddings is accomplished at least in part by performing the run-time geometry calculations to compute run-time values for the relative position bond angle vectors, the direction unit vector, and a dihedral angle for each target node; computing an updated scalar node embedding for the target node based on the computed relative position bond angle vector, the aggregated scalar messages for the target node, and the scalar node embedding for the target node; and computing an updated scalar edge embedding based on the computed dihedral angle and the scalar edge embedding, wherein
the updated molecular graph is computed based on the updated scalar edge embedding, the updated scalar node embedding, and the updated vector embedding.

18. The computerized method of claim 15, wherein, in the MPGNN processing block loop, the method further comprises:
prior to updating the vector embeddings and scalar embeddings, respectively aggregating the vector embeddings and scalar embeddings for each target node across the source nodes connected to the target node to generate a respective aggregated scalar message and aggregated vector message for the target node.

19. The computerized method of claim 13, further comprising:
during a training phase prior to the inference phase,
training the MPGNN on a training data set including multiple molecular graphs for different conformation geometries of the molecular system, and a respective ground truth value for the target molecular property for each molecular graph, the target molecular property being an energy parameter, a force parameter, or a dipole moment.

20. A computing system, comprising:
one or more processors configured to:
during an inference phase,
execute a message passing graph neural network (MPGNN) including an embedding block, one or more MPGNN processing blocks each including a respective message passing sub-block and a respective update sub-block, and an output block, wherein the message passing sub-block is configured with a vector scalar interactive message passing mechanism;
receive a molecular graph of a molecular system as input to the MPGNN, the molecular graph including nodes connected by edges, the nodes representing atoms and the edges representing interatomic bonds in the molecular system;
process the molecular graph using the embedding block to thereby produce scalar embeddings encoding scalar information describing features of the nodes and edges and vector embeddings representing geometric relationships among the nodes and edges of the molecular graph;
update, via the update sub-block, the vector embeddings based on the scalar information from the scalar embeddings and the vector embeddings;
update, via the update sub-block, the scalar embeddings based on run-time geometry calculations of the geometric relationships encoded in the vector embeddings;
compute, via the update sub-block, an updated molecular graph based on the updated scalar embeddings and updated vector embeddings for each node; and
output, via the output block, a value for a target molecular property of the molecular system determined based on the updated molecular graph.
